(19) United States
(12) Patent Application Publication
(10) Pub. No.: US 2025/0099353 A1
(43) Pub. Date: Mar. 27, 2025

(54) METHODS FOR SMOOTHING HAIR

(71) Applicant: L'OREAL, Paris (FR)

(72) Inventors: Vibha SHAH, Branchburg, NJ (US); Heather Lee, Wayne, NJ (US); Ronak Rughani, Edison, NJ (US); Lisa Chuyin Ye-Tse, Brooklyn, NJ (US); Aakash Parekh, Edison, NJ (US)

(21) Appl. No.: 18/355,480

(22) Filed: Jul. 20, 2023

(51) Int. Cl. A61K 8/41; A61Q 5/06; A61Q 5/12

(52) U.S. Cl. CPC A61K 8/41; A61Q 5/06; A61Q 5/12; A61K 2800/48; A61K 2800/594; A61K 2800/884

(57) ABSTRACT

The instant case relates to a method or routine comprising: (i) treating hair with an alkaline composition; (ii) treating hair with a conditioning composition; (iii) applying a leave on composition to the hair; and (iv) subjecting the hair to a thermal heat treatment. The alkaline composition comprises one or more alkalizing agents, such as an alkanolamine, and water. The conditioning composition comprises: (a) citric acid, a salt thereof, or a combination thereof; (b) cyclodextrin, a derivative thereof, or a combination thereof; (c) one or more cationic surfactants; and water. The leave on composition comprises: (a) one or more silicones; (b) one or more film-forming polymers; and (c) water. The hair is subjected to a thermal heat treatment, for example, treatment with a hot iron at a temperature of 150 to about 280° C. The methods improve hair fiber alignment, reduce frizz, and provide smoothness to hair.

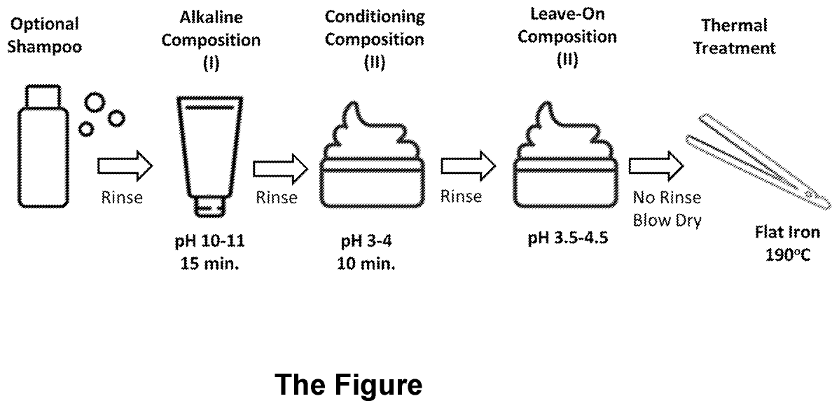

### FIELD OF THE DISCLOSURE

The present disclosure relates to methods of treating hair, for example, methods for providing hair with improved fiber alignment, frizz control, and smoothness.

### BACKGROUND

Many consumers use cosmetic and care compositions to enhance the appearance of hair, e.g., by changing the color, style, or shape of the hair and/or by imparting various cosmetic properties to hair, such as shine and conditioning. Hair can become dry or damaged for various reasons, e.g., weather exposure, poor nutrition, mechanical treatments (e.g., brushing hair), styling treatments using chemicals, dying, heat, nutrition, etc. Even cleansing products can remove hair's natural oils causing dryness, which can lead to a dull appearance, split ends, and frizz.

Chemical treatments for hair include bleaching and coloring treatments to change the color the hair. Chemical treatments also include processes to permanently change the shape and structure of the hair, for example by perming, waving, relaxing or straightening the hair. These chemical treatments change the look of hair by changing its physical structure, which inevitably causes a certain degree of damage to the hair. Environmental factors, such as salt water, sunlight, and heat, are also known to damage hair. Damaged hair is characterized by unnatural changes to the protein structure of the individual hair strands or shafts.

The popularity and usage of oils for hair treatments has increased due to their effectiveness and simplicity. Commonly used oils include olive oil, mineral oil, avocado oil, apricot kernel oil, rice bran oil, and coconut oil. However, these treatments can leave the hair feeling greasy. In addition, the effects are not usually seen after more than several hours (e.g., 8 hours) of treatment and several treatments are usually required, making it time consuming and labor intensive.

Damage to hair results in split ends, dryness, hair that is easily broken, and hair that becomes “frizzy” and unmanageable. Because the visible portion of hair is dead, it has no ability to regenerate itself. There are numerous over the counter and salon treatments that purport to repair damaged hair. These include conditioners, hot oil treatments, hydrolyzed proteins, vitamin formulations, and exotic fruit, leaf, or root extracts. These treatments, however, provide only limited improvement to the hair. Therefore, hair treatment technologies that can straighten, relax, or style the hair without chemically damaging the hair are desired.

There is still a need for providing improved manageability of hair, for example, improved hair alignment, reduced unwanted volume (especially reduced frizz), and increased shine.

### SUMMARY OF THE DISCLOSURE

The instant disclosure is drawn to methods for improving the look, feel, and style of hair. In particular, the methods improve fiber alignment, reduce frizz, and impart smoothness. The treated hair is soft, shiny, and has a healthy appearance. The methods involve a sequence of treatments that ultimately form a smooth coating on the surface of the hair, which is surprisingly durable. The coating and desirable cosmetic properties imparted to the hair are long-lasting, resistant to humidity, and maintained even after washing the hair. In addition, the methods are gentle and do not damage the hair.

The hair is treated in multiple steps, each step treating the hair with a different hair treatment composition, which in aggregate results in surprisingly robust and long-lasting benefits. Simply, the hair is first treated with an alkaline composition, followed by treatment with a conditioning composition, and finally treated with a leave-on composition. The alkaline composition and the conditioning compositions are usually individually rinsed from the hair after treating the hair.

Initially, the hair is treated with an alkaline composition, for example, an alkaline composition having a pH of about 9 to about 12. The alkaline composition helps open the hair cuticle facilitate straightening. As the same suggests, the alkaline composition includes one or more alkalizing agents. In various embodiments, it is preferably for at least one of the one or more alkalizing agents to be an alkanolamine. Nonlimiting examples of alkanolamines include ethanolamine, diethanolamine, triethanolamine, monopropanolamine, isopropanolamine, dipropanolamine, tripropanolamine, 2-amino-2-methyl-1,3-propanediol and 2-amino-2-hydroxymethyl-1,3-propanediol. Nonlimiting examples of additional alkalizing agents include ammonia, taurine, guanidine and guanidium salts, hydroxide-containing compounds, and combinations thereof. Nonlimiting examples of hydroxide-containing compounds include alkali metal hydroxides, alkaline-earth metal hydroxides, and mixtures thereof, for example, sodium hydroxide, potassium hydroxide, lithium hydroxide, calcium hydroxide, magnesium hydroxide, barium hydroxide, strontium hydroxide, and mixtures thereof.

The alkaline composition is typically an aqueous composition and thus, contains a significant amount of water. In addition, the alkalizing composition optionally include one or more fatty compounds, including one or more fatty alcohols, one or more water soluble solvents, one or more nonionic surfactants or emulsifiers, one or more thickening agents, for example, one or more polysaccharide thickening agents, one or more miscellaneous ingredients, or combinations thereof.

The alkaline composition is applied to the hair and remains on the hair for a period of time, e.g., a period of time sufficient for the alkaline composition to open the hair cuticle and facilitate straightening. For example, the alkaline composition may remain on the hair for about 1 minute to about 60 minutes, about 5 minutes to about 45 minutes, or amount 10 minutes to about 30 minutes. After treating the hair for the period of time, the alkaline composition is typically rinsed from the hair with water.

The hair is subsequently treated with a condition composition, which is also allowed to remain on the hair for a period of time. The conditioning composition includes a unique combination of: (a) citric acid, a salt thereof, or a combination thereof; and (b) a cyclodextrin, a derivative thereof, or a combination thereof. The conditioning composition improves fiber alignment, reduces frizz, and imparts smoothness to the hair. The conditioning composition includes a unique combination of a components that form a smooth coating on the surface of the hair that is particularly durable and long lasting. This is achieved, at least in party, by including a unique combination of: (a) citric acid, a salt thereof, or a combination thereof; and (b) a cyclodextrin, a derivative thereof, or a combination thereof. For example, a combined total amount of the citric acid, salt thereof, or combination thereof of (a) and the cyclodextrin, derivative thereof, or combination thereof of (b) is about 2 to about 12 wt. %, based on a total weight of the conditioning composition.

In addition, the conditioning compositions typically include one or more cationic surfactants. Cationic surfactants, such as cetrimonium chloride and stearamidopropyl dimethylamine, have a positive charge on their head group. The structure of the molecules can vary, but is typically a fatty, acid-derived, hydrophobic tail with a nitrogen-containing head group. The nitrogen-containing group can be, for example, a tertiary or quaternary amine. Typically, these surfactants are either alkyl amine salts or alkyl quaternary ammonium salts. The head groups of these cationic surfactants experience electrostatic attraction to hair and adsorb onto the surface of the hair. The hydrophobic portion of the surfactant molecule lies flat along the surface of the hair, forming a film that smooths the cuticle, resulting in a reduction of static, reduction of combing forces, increase in pleasing tactile feel, and a decrease in tangle formation.

The conditioning composition includes water and optionally one or more additional components. For example, the conditioning compositions optionally include one or more nonionic surfactants, one or more fatty alcohols, one or more amino-functionalized silicones, one or more water soluble solvents, one or more fatty compounds other than the one or more fatty alcohols, one or more cationic polymers, one or more miscellaneous ingredients, or a combination thereof.

The conditioning composition is applied to the hair and remains on the hair for a period of time, e.g., a period of time sufficient to impart its conditioning effects to the hair. For example, the conditioning composition may remain on the hair for about 1 minute to about 60 minutes, about 2 minutes to about 30 minutes, or amount 5 minutes to about 20 minutes. After treating the hair with the conditioning composition for a period of time, the conditioning composition is typically rinsed from the hair with water.

After treatment with the conditioning composition, a leave-on composition is applied to the hair. As the name suggest, a “leave-on” composition is not rinsed from the hair prior to styling the hair. The leave-on composition includes one or more silicones, which are helpful for protecting the hair from damage by heat and provides slip and shine to the hair. In various embodiments, an amino-functionalized silicone is preferred. In addition, the leave-on composition usually includes one or more film-forming polymers and water. Nonlimiting film-forming polymers that can be used include nonionic associative polymeric thickeners. As the name suggests, such polymers are nonionic and can provide thickening properties to the composition. In addition to water, the leave-on composition may optionally further include one or more fatty compounds, one or more polysaccharide thickening agents, for example, sclerotium gum, one or more water soluble solvents, one or more miscellaneous ingredients, or a combination thereof.

After the leave-on composition has been applied to the hair, preferably uniformly applied throughout the hair, the hair can be blow dried, or allowed to dry naturally, and subjected to a thermal treatment (treated with heat). For example, the hair can be treated with a hot iron, in particular, a flat iron. Typically, the hot iron is passed over the hair at least once, at least twice, at least three times, or more. The hot iron is preferably at a temperature of about 150° C. to about 300° C., preferably 150° C. to about 250° C., more preferably about 180° C. to about 230° C.

Hair treated with the methods described above exhibits improved fiber alignment, reduced frizz, smoothness, and shine. Despite the formation of a film on the hair, the hair is soft, shiny, and has a healthy appearance. The desirable cosmetic properties imparted to the hair are long-lasting, resistant to humidity, and maintained even after washing the hair.

### BRIEF DESCRIPTION OF THE DRAWINGS

Implementation of the present technology is described, by way of example only, with reference to the attached FIGURE, wherein:

THE FIGURE outlines the methods and steps of the methods described throughout the instant disclosure.

The various aspects are not limited to the arrangements, composition, and instrumentality shown in the drawings.

### DETAILED DESCRIPTION OF THE DISCLOSURE

Methods for improving fiber alignment, reducing frizz, impart smoothness, and straightening hair are described in detail below. The compositions used in the methods include unique combinations of a particular components that ultimately result in the formation of a smooth coating on the surface of the hair. The smooth coating is surprisingly durable, resistant to humidity, and withstands washing.

In various embodiments, the method includes:
(I) applying an alkaline composition having a pH of about 9 to about 12 to the hair, allowing the composition to remain on the hair for a period of time, and rinsing the alkaline composition from the hair; the alkaline composition comprising:
   (a) one or more alkalizing agents comprising:
      (a)(i) one or more alkanolamines; and
      (a)(ii) optionally, one or more additional alkalizing agents; and
   (b) water;
(II) after rinsing the alkaline composition from the hair, applying a conditioning composition to the hair, allowing the conditioning composition to remain on the hair for a period of time, and rinsing the conditioning composition from the hair; the conditioning composition comprising:
   (a) citric acid, a salt thereof, or a combination thereof;
   (b) cyclodextrin, a derivative thereof, or a combination thereof;
      wherein a combined total amount of (a) and (b) is about 2 to about 8 wt. %;
   (c) one or more cationic surfactants; and
   (h) water; and
(III) after rinsing the conditioning composition from the hair, applying a leave-on composition to the hair, the leave-on hair composition comprising:
   (a) one or more silicones;
   (b) one or more film-forming polymers; and
   (c) water.

The hair is treated in multiple steps, each step treating the hair with a different hair treatment composition, which in aggregate results in surprisingly robust and long-lasting benefits. Simply, the hair is first treated with an alkaline composition, followed by treatment with a conditioning composition, and finally treated with a leave-on composition. The alkaline composition and the conditioning compositions are usually individually rinsed from the hair after treating the hair.

Initially, the hair is treated with an alkaline composition, for example, an alkaline composition having a pH of about 9 to about 12. The alkaline composition helps open the hair cuticle facilitate straightening. As the same suggests, the alkaline composition includes one or more alkalizing agents. In various embodiments, it is preferably for at least one of the one or more alkalizing agents to be an alkanolamine. Nonlimiting examples of alkanolamines include ethanolamine, diethanolamine, triethanolamine, monopropanolamine, isopropanolamine, dipropanolamine, tripropanolamine, 2-amino-2-methyl-1,3-propanediol and 2-amino-2-hydroxymethyl-1,3-propanediol, and mixtures thereof. Nonlimiting examples of additional alkalizing agents include ammonia, taurine, guanidine and guanidium salts, hydroxide-containing compounds, and combinations thereof. Nonlimiting examples of hydroxide-containing compounds include alkali metal hydroxides, alkaline-earth metal hydroxides, and mixtures thereof, for example, sodium hydroxide, potassium hydroxide, lithium hydroxide, calcium hydroxide, magnesium hydroxide, barium hydroxide, strontium hydroxide, and mixtures thereof.

The alkaline composition is typically an aqueous composition and thus, contains a significant amount of water. In addition, the alkalizing composition optionally include one or more fatty compounds, including one or more fatty alcohols, one or more water soluble solvents, one or more nonionic surfactants or emulsifiers, one or more thickening agents, for example, one or more polysaccharide thickening agents, one or more miscellaneous ingredients, or combinations thereof.

The alkaline composition is applied to the hair and remains on the hair for a period of time, e.g., a period of time sufficient for the alkaline composition to open the hair cuticle and facilitate straightening. For example, the alkaline composition may remain on the hair for about 1 minute to about 60 minutes, about 5 minutes to about 45 minutes, or amount 10 minutes to about 30 minutes. After treating the hair for the period of time, the alkaline composition is typically rinsed from the hair with water.

The hair is subsequently treated with a condition composition, which is also allowed to remain on the hair for a period of time. The conditioning composition includes a unique combination of: (a) citric acid, a salt thereof, or a combination thereof; and (b) a cyclodextrin, a derivative thereof, or a combination thereof. The conditioning composition improves fiber alignment, reduces frizz, and imparts smoothness to the hair. The conditioning composition includes a unique combination of a components that form a smooth coating on the surface of the hair that is particularly durable and long lasting. This is achieved, at least in party, by including a unique combination of: (a) citric acid, a salt thereof, or a combination thereof; and (b) a cyclodextrin, a derivative thereof, or a combination thereof. For example, a combined total amount of the citric acid, salt thereof, or combination thereof of (a) and the cyclodextrin, derivative thereof, or combination thereof of (b) is about 2 to about 12 wt. %, based on a total weight of the conditioning composition.

In addition, the conditioning compositions typically include one or more cationic surfactants. Cationic surfactants, such as cetrimonium chloride and stearamidopropyl dimethylamine, have a positive charge on their head group. The structure of the molecules can vary, but is typically a fatty, acid-derived, hydrophobic tail with a nitrogen-containing head group. The nitrogen-containing group can be, for example, a tertiary or quaternary amine. Typically, these surfactants are either alkyl amine salts or alkyl quaternary ammonium salts. The head groups of these cationic surfactants experience electrostatic attraction to hair and adsorb onto the surface of the hair. The hydrophobic portion of the surfactant molecule lies flat along the surface of the hair, forming a film that smooths the cuticle, resulting in a reduction of static, reduction of combing forces, increase in pleasing tactile feel, and a decrease in tangle formation.

The conditioning composition includes water and optionally one or more additional components. For example, the conditioning compositions optionally include one or more nonionic surfactants, one or more fatty alcohols, one or more amino-functionalized silicones, one or more water soluble solvents, one or more fatty compounds other than the one or more fatty alcohols, one or more cationic polymers, one or more miscellaneous ingredients, or a combination thereof.

The conditioning composition is applied to the hair and remains on the hair for a period of time, e.g., a period of time sufficient to impart its conditioning effects to the hair. For example, the conditioning composition may remain on the hair for about 1 minute to about 60 minutes, about 2 minutes to about 30 minutes, or amount 5 minutes to about 20 minutes. After treating the hair with the conditioning composition for a period of time, the conditioning composition is typically rinsed from the hair with water.

After treatment with the conditioning composition, a leave-on composition is applied to the hair. As the name suggest, a “leave-on” composition is not rinsed from the hair prior to styling the hair. The leave-on composition includes one or more silicones, which are helpful for protecting the hair from damage by heat and provides slip and shine to the hair. In various embodiments, an amino-functionalized silicone is preferred. In addition, the leave-on composition usually includes one or more film-forming polymers and water. Nonlimiting film-forming polymers that can be used include nonionic associative polymeric thickeners. As the name suggests, such polymers are nonionic and can provide thickening properties to the composition. In addition to water, the leave-on composition may optionally further include one or more fatty compounds, one or more polysaccharide thickening agents, for example, sclerotium gum, one or more water soluble solvents, one or more miscellaneous ingredients, or a combination thereof.

After the leave-on composition has been applied to the hair, preferably uniformly applied throughout the hair, the hair can be blow dried, or allowed to dry naturally, and subjected to a thermal treatment (treated with heat). For example, the hair can be treated with a hot iron, in particular, a flat iron. Typically, the hot iron is passed over the hair at least once, at least twice, at least three times, or more. The hot iron is preferably at a temperature of about 150° C. to about 300° C., preferably 150° C. to about 250° C., more preferably about 180° C. to about 230° C.

More details relating to each of the compositions useable in the methods of the instant disclosure and details relating to their use in the methods of the instant disclosure are set forth below.

### Alkaline Composition

In various embodiments, the alkaline compositions of (I) include:
(a) one or more alkalizing agents comprising:
   (a)(i) one or more alkanolamines; and
   (a)(ii) optionally, one or more additional alkalizing agents; and
(b) water;
(c) about 2 to about 20 wt. % of one or more non-silicone based fatty compounds comprising:
   (c)(i) about 2 to about 12 wt. % of one or more fatty alcohols; and
   (c)(ii) optionally, about 1 to about 10 wt. % of one or more additional fatty compounds.

In further embodiments, the alkaline compositions of (1) may further include:
(d) optionally, about 1 to about 20 wt. % of one or more water soluble solvents; and/or
(e) optionally, about 1 to about 8 wt. % of one or more nonionic surfactants or emulsifiers; and/or
(f) optionally, one or more polysaccharide thickening agents;
   wherein all weight percentages are based on a total weight of the alkaline composition.

In various embodiments, the alkaline composition may optionally include one or more silicones, one or more miscellaneous ingredients, or a combination thereof.

The pH of the alkaline composition will vary but as the name suggest, the pH is alkaline (above 7). In various embodiments, the pH of the alkaline composition is about 9 to about 12. In further embodiments, the pH of the alkaline composition is about 9 to about 11, about 9 to about 10, about 10 to about 12, about 10 to about 11, about 11 to about 12, or about 9, about 9.5, about 10, about 10.5, about 11, about 11.5, or about 12.

### (I)(a) Alkalizing Agents

Many alkalizing agents are known and can be used in the alkaline composition. Non-limiting examples of alkalizing agents include alkali metal hydroxides (e.g., lithium hydroxide, sodium hydroxide, potassium hydroxide, magnesium hydroxide, magnesium oxide), alkaline-earth metal hydroxides (e.g., calcium hydroxide), alkali metal silicates, (e.g., sodium silicate, lithium silicate, and potassium silicate, magnesium silicate, aluminum silicate, fluorphlogopite), alkali metal carbonates (e.g., lithium carbonate, sodium carbonate, potassium carbonate, sodium bicarbonate), alkaline-earth metal carbonates (e.g., calcium carbonate), organic carbonates (e.g., guanidine carbonate), basic amino acids (arginine, lysine, histidine), and their polymers (poly arginine, poly lysine, etc.), organic amines, such as alkanolamines (e.g., monoethanolamine, diethanolamine, triethanolamine, aminomethyl propanol), ammonium hydroxide, and a mixture thereof.

### (I)(a)(i) Organic Amines Including Alkanolamines

Organic amines include, but are not limited to, those having one or two primary, secondary, or tertiary amine functions, and at least one linear or branched C1-C8 alkyl groups bearing at least one hydroxyl radical. Organic amines include cyclic amines and other cyclic compounds, saturated or unsaturated, having one or more nitrogen atoms within the ring, and mixtures thereof. The organic amines may be chosen from those having a pKb at 25° C. of less than 12, such as less than 10 or such as less than 6. It should be noted that this is the pKb corresponding to the function of highest basicity. Nonlimiting examples includes alkanolamines such as mono-, di- or trialkanolamines, comprising one to three identical or different C1-C4 hydroxyalkyl radicals, ethylamines, ethyleneamines, quinoline, aniline and cyclic amines, such as pyrroline, pyrrole, pyrrolidine, imidazole, imidazolidine, imidazolidinine, morpholine, pyridine, piperidine, pyrimidine, piperazine, triazine and derivatives thereof. Non-limiting examples of alkanolamines include ethanolamine, diethanolamine, triethanolamine, monopropanolamine, isopropanolamine, dipropanolamine, tripropanolamine, 2-amino-2-methyl-1,3-propanediol and 2-amino-2-hydroxymethyl-1,3-propanediol, and mixtures thereof, triisopropanolamine, 2-amino-2-methyl-1,3-propanediol, 3-amino-1,2-propanediol, 3-dimethylamino-1,2-propanediol, 2-amino-2-methyl-1-propanol (aminomethyl propanol or AMP), tris(hydroxymethylamino)methane, tetrahydroxypropyl ethylenediamine, tromethamine, a mixture thereof. In a preferred embodiment, the one or more alkanolamines are selected from ethanolamine, diethanolamine, triethanolamine, aminomethyl propanol, or a combination thereof.

In instances, the organic amines may be organic amines of heterocyclic type. Besides histidine that has already been mentioned in the amino acids, non-limiting mention may also be made of pyridine, piperidine, imidazole, 1,2,4-triazole, tetrazole, and benzimidazole. In some cases, the organic amines may be amino acid dipeptides. Amino acid dipeptides that may be used in the present disclosure include but not limited to carnosine, anserine, and baleine.

The organic amine may be in salt form. The term “organic amine salt,” as used herein, means organic or mineral salts of an organic amine as described above. As a non-limiting example, the organic salts may be chosen from the salts of organic acids, such as citrates, lactates, glycolates, gluconates, acetates, propionates, fumarates, oxalates and tartrates. In one embodiment, the organic salt is trisodium citrate. Further as a non-limiting example, the mineral salts may be chosen from hydrohalides (for example hydrochlorides), carbonates, hydrogen carbonates, sulfates, hydrogen phosphates, and phosphates. The ammonium salts that may be used according to the present disclosure may be chosen from the following acid salts: carbonate, bicarbonate. For instance, the salt is the carbonate, such as ammonium carbonate.

The alkali metal phosphates and carbonates that may be used are, for example, sodium phosphate, potassium phosphate, sodium carbonate, sodium bicarbonate, potassium carbonate, potassium bicarbonate, and their derivatives.

In preferred embodiments, at least one of the one or more alkalizing agents includes or is selected from one or more alkanolamines, for example (without limitation) one or more alkanolamines are selected from ethanolamine, diethanolamine, triethanolamine, aminomethyl propanol, or a combination thereof. The total amount of the one or more alkanolamines in the alkaline composition will vary depending on a variety of factors, for example, the target pH. Nonetheless, in various embodiments, the alkaline composition includes about 1 to about 15 wt. % of one or more alkanolamines, based on a total weight of the alkaline composition. In further embodiments, the alkaline composition includes about 1 to about 12 wt. %, about 1 to about 10 wt. %, about 1 to about 8 wt. %, about 2 to about 15 wt. %, about 2 to about 12 wt. %, about 2 to about 10 wt. %, about 2 to about 8 wt. %, about 3 to about 15 wt. %, about 3 to about 12 wt. %, about 3 to about 10 wt. %, about 3 to about 8 wt. %, about 5 to about 15 wt. %, about 5 to about 12 wt. %, about 5 to about 10 wt. %, or about 5 to about 8 wt. %, based on a total weight of the alkaline composition.

### (I)(a)(ii) Hydroxide Alkalizing Agents

Alkalizing agents may also be chosen from hydroxide compounds (also called hydroxide bases) such as alkali metal hydroxides, alkaline-earth metal hydroxides, transition metal hydroxides, quaternary ammonium hydroxides, organic hydroxides, and mixtures thereof. Suitable examples are ammonium hydroxide, sodium hydroxide, potassium hydroxide, lithium hydroxide, rubidium hydroxide, cesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminum hydroxide, guanidinium hydroxide and mixtures thereof.

In a preferred embodiment, at least one of the one or more alkalizing agent includes or is selected from one or more hydroxide bases. The total amount of the one or more hydroxide bases will vary depending on a variety of factors, for example, the target pH. Nonetheless, in various embodiments, the alkaline composition includes about 0.1 to about 8 wt. % of the one or more hydroxide bases. In further embodiments, the alkaline composition includes about 0.1 to about 5 wt. %, 0.1 to about 3 wt. %, about 0.1 to about 2 wt. %, about 0.1 to about 1 wt. %, about 0.5 to about 8 wt. %, about 0.5 to about 5 wt. %, about 0.5 to about 3 wt. %, about 0.5 to about 2 wt. %, or about 0.5 to about 1 wt. %, based on a total weight of the alkaline composition.

### (I)(a)(iii) Amino Acids

As non-limiting examples, the amino acids that may be used may be of natural or synthetic origin, in L, D, or racemic form, and comprise at least one acid function chosen from, for instance, carboxylic acid, sulfonic acid, phosphonic acid, and phosphoric acid functions. The amino acids may be in their neutral or ionic form. Polymeric forms are also useful, such as poly arginine, poly lysine, etc. Additional non-limiting examples include basic amino acids comprising an additional amine function optionally included in a ring or in a ureido function. In some cases, amino acids that may be used include, but are not limited to, aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine, ornithine, citrulline, and valine.

In preferred embodiments, at least one of the one or more alkalizing agent includes or is selected from one or more amino acids, for example, taurine. The total amount of the one or more amino acids will vary depending on a variety of factors. Nonetheless, in various embodiments, the alkaline composition includes about 0.1 to about 10 wt. % of one or more amino acids, based on a total weight of the alkaline composition. In further embodiments, the alkaline composition includes about 0.1 to about 8 wt. %, about 0.1 to about 5 wt. %, about 0.5 to about 10 wt. %, about 0.5 to about 8 wt. %, about 0.5 to about 5 wt. %, about 1 to about 10 wt. %, about 1 to about 8 wt. %, or about 1 to about 5 wt. %, based on a total weight of the alkaline composition.

### (I)(a)(iv) Guanidine Amines

The organic amines may also be chosen from compounds comprising a guanidine function. Organic amines of this type include, besides arginine that has already been mentioned as an amino acid, creatine, creatinine, 1,1-dimethylguanidine, 1,1-diethylguanidine, glycocyamine, metformin, agmatine, N-amidinoalanine, 3-guanidinopropionic acid, 4-guanidinobutyric acid, and 2-([amino(imino)methyl]amino)ethane-1-sulfonic acid.

In various embodiments, the alkaline composition includes: (1) one or more alkanolamine; and (2) one or more amino acids, one or more hydroxide bases, or a mixture thereof. For example, the alkaline composition may include one or more alkanolamines selected from selected from ethanolamine, diethanolamine, triethanolamine, aminomethyl propanol, or a combination thereof, preferably ethanolamine; and one or more amino acids, preferably taurine; and one or more hydroxide bases, preferably selected from ammonium hydroxide, sodium hydroxide, potassium hydroxide, lithium hydroxide, rubidium hydroxide, cesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminum hydroxide, guanidinium hydroxide and mixtures thereof, preferably sodium hydroxide.

The total amount of the one or more alkalizing agents in the alkaline composition will vary depending on a variety of factors, for example, the target pH. Nonetheless, in various embodiments, the alkaline compositions include about 1 to about 25 wt. % of the one or more alkalizing compounds, based on a total weight of the alkaline composition. In further embodiments, the alkaline composition includes about 1 to about 20 wt. %, about 1 to about 15 wt. %, about 1 to about 12 wt. %, about 2 to about 20 wt. %, about 2 to about 15 wt. %, about 2 to about 12 wt. %, about 3 to about 20 wt. %, about 3 to about 15 wt. %, about 3 to about 12 wt. %, about 5 to about 20 wt. %, about 5 to about 15 wt. %, about 5 to about 12 wt. %, about 8 to about 20 wt. %, about 8 to about 15 wt. %, or about about 8 to about 12 wt. %, based on a total weight of the alkaline composition.

### (I)(b) Water

The total amount of water in the alkaline composition will vary depending on a variety of factors. Nonetheless, in various embodiments, the alkaline compositions include about 50 to about 90 wt. % of water. In further embodiments, the alkaline compositions include about 50 to about 80 wt. %, about 50 to about 75 wt. %, about 50 to about 70 wt. %, about 55 to about 90 wt. %, about 55 to about 80 wt. %, about 55 to about 75 wt. %, about 55 to about 70 wt. %, about 60 to about 90 wt. %, about 60 to about 80 wt. %, about 60 to about 75 wt. %, or about 60 to about 70 wt. %, based on a total weight of the alkaline composition.

### (I)(c) Non-Silicone Based Fatty Compounds

The term “non-silicone based fatty compound” can also be set forth as simply, “fatty compound.” The term “fatty substance” means an organic compound without silicone that is insoluble in water at ordinary temperature (25° C.) and at atmospheric pressure (760 mmHg), i.e. which has a solubility of less than 5%, preferably less than 1% and even more preferentially less than 0.1%. They have in their structure a hydrocarbon-based chain containing at least 6 carbon atoms.

More particularly, the one or more non-silicone-based fatty compounds may be selected from C6-C16 hydrocarbons, hydrocarbons containing more than 16 carbon atoms, non-silicone oils of animal origin, plant oils of triglyceride type, synthetic triglycerides, fluoro oils, fatty alcohols, non-salified fatty acids, fatty acid and/or fatty alcohol esters other than triglycerides and plant waxes, non-silicone waxes and silicones, and mixtures thereof.

Fatty alcohols, fatty esters and fatty acids more particularly contain one or more linear or branched, saturated or unsaturated hydrocarbon-based groups comprising 6 to 30 carbon atoms, which are optionally substituted, in particular, with one or more (in particular 1 to 4) hydroxyl groups. If they are unsaturated, these compounds may comprise one to three conjugated or unconjugated carbon-carbon double bonds.

As regards the C6-C16 hydrocarbons, they are linear, branched or optionally cyclic, and are preferably alkanes. Examples that may be mentioned include hexane, dodecane and isoparaffins such as isohexadecane and isodecane.

A hydrocarbon-based oil of animal origin that may be mentioned is perhydrosqualene.

The triglyceride oils of plant or synthetic origin are preferably chosen from liquid fatty acid triglycerides comprising from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearineries Dubois or those sold under the names Miglyol® 810, 812 and 818 by the company Dynamit Nobel, jojoba oil and shea butter oil.

The linear or branched hydrocarbons of mineral or synthetic origin containing more than 16 carbon atoms are preferably chosen from liquid paraffins, petroleum jelly, liquid petroleum jelly, polydecenes and hydrogenated polyisobutene such as Parleam®.

The fluoro oils may be chosen from perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxybutane and nonafluoroethoxyisobutane; perfluoromorpholine derivatives such as 4-trifluoromethyl perfluoromorpholine sold under the name PF 5052® by the company 3M.

The fatty alcohols that may be used in the cosmetic composition of step a) are saturated or unsaturated, linear or branched alcohols comprising from 6 to 30 carbon atoms and more particularly from 8 to 30 carbon atoms, among which mention may be made, for example, of cetyl alcohol, stearyl alcohol and the mixture thereof (cetylstearyl alcohol or cetearyl alcohol), octyldodecanol, 2-butyloctanol, 2-hexyldecanol, 2-undecylpentadecanol, oleyl alcohol or linoleyl alcohol.

The non-salified fatty acids that may be used in the cosmetic composition of step a) may be saturated or unsaturated carboxylic acids comprising from 6 to 30 carbon atoms and in particular from 9 to 30 carbon atoms. They are more particularly chosen from myristic acid, palmitic acid, stearic acid, behenic acid, oleic acid, linoleic acid, linolenic acid and isostearic acid.

These acids are not salified. This means that they are introduced in the form of free acids and that the composition does not comprise any alkaline agent leading to their salification.

The esters of fatty acids and/or of fatty alcohols, advantageously different from the triglycerides mentioned above, which may be used in the cosmetic composition used in step a) are esters of saturated or unsaturated, linear or branched C1-C26 aliphatic mono- or polyacids and of saturated or unsaturated, linear or branched C1-C26 aliphatic mono- or polyalcohols, the total carbon number of the esters more particularly being greater than or equal to 10. Among the monoesters, mention may be made of dihydroabietyl behenate; octyldodecyl behenate; isocetyl behenate; cetyl lactate; C12-C15 alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate.

Still within the context of this variant, esters of C4-C22 dicarboxylic or tricarboxylic acids and of C1-C22 alcohols and esters of mono-, di- or tricarboxylic acids and of C2-C26 di-, tri-, tetra- or pentahydroxy alcohols may also be used.

Mention may be made in particular of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate, tridecyl erucate; triisopropyl citrate; triisostearyl citrate; glyceryl trilactate; glyceryl trioctanoate; trioctyldodecyl citrate; trioleyl citrate, propylene glycol dioctanoate; neopentyl glycol diheptanoate; diethylene glycol diisononanoate; and polyethylene glycol distearates.

Among the esters mentioned above, it is preferred to use ethyl, isopropyl, myristyl, cetyl or stearyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate or cetyl octanoate.

The esters according to this variant may also be chosen from monoesters, diesters, triesters, tetraesters and polyesters, and mixtures thereof. These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters. More particularly, use is made of monoesters and diesters and in particular mono- or di-oleate, -stearate, -behenate, -oleopalmitate, -linoleate, -linolenate or -oleostearate of sucrose, glucose or methylglucose.

The non-silicone wax(es) that may be used in the cosmetic composition used in step a) are chosen especially from carnauba wax, candelilla wax, esparto grass wax, hydrocarbon waxes including paraffin wax, ozokerite and microcrystalline wax, plant waxes such as olive wax, rice wax, hydrogenated jojoba wax or the absolute waxes of flowers such as the essential wax of blackcurrant blossom sold by the company Bertin (France), animal waxes, for instance beeswaxes or modified beeswaxes (cerabellina); other waxes or waxy starting materials that may be used according to the invention are especially marine waxes such as the product sold by the company Sophim under the reference M82, and polyethylene waxes or polyolefin waxes in general.

In a preferred embodiment, the one or more non-silicone-based fatty compounds are selected from oils, waxes, linear or branched alkanes, fatty esters, esters of fatty acids, esters of fatty alcohols, cetyl esters, triglycerides, or a mixture thereof.

### (I)(c)(i) Fatty Alcohols

In various embodiments, it is preferable if at least one of the one or non-silicone fatty compounds includes one or more fatty alcohols. The term “fatty alcohol” means an alcohol comprising at least one hydroxyl group (OH), and comprising at least 8 carbon atoms, and which is neither oxyalkylenated (in particular neither oxyethylenated nor oxypropylenated) nor glycerolated. The fatty alcohols can be represented by: R—OH, wherein R denotes a saturated (alkyl) or unsaturated (alkenyl) group, linear or branched, comprising from 8 to 40 carbon atoms, preferably 10 to 30 carbon atoms, more preferably 12 to 24 carbon atoms, and even more preferably 14 to 22 carbon atoms.

The fatty alcohol(s) may be liquid or solid. In some instances, it is preferable that the cosmetic compositions include at least one solid fatty alcohol. The solid fatty alcohols that can be used include those that are solid at ambient temperature and at atmospheric pressure (25° C., 780 mmHg), and are insoluble in water, that is to say they have a water solubility of less than 1% by weight, preferably less than 0.5% by weight, at 25° C., 1 atm.

The solid fatty alcohols may be represented by: R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

Non-limiting examples of useful fatty alcohols include lauryl alcohol or lauryl alcohol (1-dodecanol); myristic or myristyl alcohol (1-tetradecanol); cetyl alcohol (1-hexadecanol); stearyl alcohol (1-octadecanol); arachidyl alcohol (1-eicosanol); behenyl alcohol (1-docosanol); lignoceryl alcohol (1-tetracosanol); ceryl alcohol (1-hexacosanol); montanyl alcohol (1-octacosanol); myricylic alcohol (1-triacontanol), and mixtures thereof.

In certain embodiments, the one or more fatty alcohols have from 12 to 24 carbon atoms. Specific nonlimiting examples include cetyl alcohol, stearyl alcohol, cetearyl alcohol, behenyl alcohol, lauryl alcohol, myristic or myristyl alcohol, arachidyl alcohol, lignoceryl alcohol, or mixtures thereof.

Preferably, the cosmetic composition includes one or more solid fatty alcohols, for example, chosen from cetyl alcohol, stearyl alcohol, behenyl alcohol and mixtures thereof, preferably cetyl alcohol, behenyl alcohol, cetearyl alcohol, and mixtures thereof.

The liquid fatty alcohols, in particular those containing C10-C34, preferably have branched carbon chains and/or have one or more, preferably 1 to 3 double bonds. They are preferably branched and/or unsaturated (C═C double bond) and contain from 12 to 40 carbon atoms.

The liquid fatty alcohols may be represented by: R—OH, wherein R denotes a C12-C24 branched alkyl group or an alkenyl group (comprising at least one C12-C24 double bond C═C), R being optionally substituted by a or more hydroxy groups. Preferably, the liquid fatty alcohol is a branched saturated alcohol. Preferably, R does not contain a hydroxyl group. These include oleic alcohol, linoleic alcohol, linolenic alcohol, isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol and mixtures thereof. Preferably, the liquid fatty alcohol is 2-octyl-1-dodecanol.

In some instances, the cosmetic compositions include one or more fatty alcohols selected from decyl alcohol, undecyl alcohol, dodecyl, myristyl, cetyl alcohol, stearyl alcohol, cetearyl alcohol, isostearyl alcohol, isocetyl alcohol, behenyl alcohol, linalool, oleyl alcohol, myricyl alcohol and a mixture thereof. In some instances, the cosmetic compositions preferably include cetyl alcohol, behenyl alcohol, or cetearyl alcohol, or a combination thereof.

The total amount of the one or more fatty alcohols in the alkaline composition will vary depending on a variety of factors. Nonetheless, in various embodiments, the alkaline composition includes about 0.1 to about 10 wt. % of the one or more fatty alcohols, based on a total weight of the alkaline composition. In further embodiments, the alkaline compositions include about 0.1 to about 8 wt. %, about 0.1 to about 5 wt. %, about 0.5 to about 8 wt. %, about 0.5 to about 10 wt. %, about 0.5 to about 5 wt. %, about 1 to about 10 wt. %, about 1 to about 8 wt. %, about 1 to about 5 wt. %, about 2 to about 10 wt. %, about 2 to about 8 wt. %, about 2 to about 5 wt. % of the one or more fatty alcohols, based on a total weight of the alkaline composition.

### (I)(c)(ii) Additional Fatty Compounds

In various embodiments, it is preferable if at least one of the one or more non-silicone fatty compounds includes one or more additional non-silicone fatty compounds (or simply additional fatty compounds). An additional fatty compound is a fatty compound other than a fatty alcohol and includes those set forth above except for the fatty alcohols. In various embodiments, the one or more additional fatty compounds include or are selected from C6-C16 hydrocarbons, being linear, branched or optionally cyclic, and are preferably alkanes. Examples that may be mentioned include hexane, dodecane and isoparaffins such as isohexadecane and isodecane, C13-C16 isoalkane or C13-C16 isoparaffin, or a combination thereof.

The total amount of the one or more additional fatty compounds will vary depending on a variety of factors. Nonetheless, in various embodiments, the total amount of the one or more additional fatty compounds is from about 0.1 to about 18 wt. %, based on a total weight of the alkaline composition. In further embodiments, the alkaline composition includes about 0.1 to about 15 wt. %, about 0.1 to about 12 wt. %, about 0.1 to about 10 wt. %, about 0.1 to about 8 wt. %, about 1 to about 18 wt. %, about 1 to about 12 wt. %, about 1 to about 10 wt. %, about 1 to about 12 wt. %, about 1 to about 8 wt. %, about 3 to about 18 wt. %, about 3 to about 15 wt. %, about 3 to about 12 wt. %, about 3 to about 10 wt. %, or about 3 to about 8 wt. %, based on a total weight of the alkaline composition.

The total amount of the one or more non-silicone based fatty compounds (all fatty compounds including fatty alcohol, additional fatty compounds, or combination thereof) will vary depending on a variety of factors. Nonetheless, in various embodiments, the alkaline composition includes about 1 to about 20 wt. % of the one or more fatty compounds. In further embodiments, the alkaline compositions include about 1 to about 15 wt. %, about 1 to about 12 wt. %, about 1 to about 10 wt. %, about 2 to about 20 wt. %, about 2 to about 15 wt. %, about 2 to about 12 wt. %, about 2 to about 10 wt. %, about 5 to about 20 wt. %, about 5 to about 15 wt. %, about 5 to about 12 wt. %, or about 5 to about 10 wt. % of the one or more non-silicone based fatty compounds.

### (I)(d) Water Soluble Solvents

The term “water soluble organic solvent” is interchangeable with the terms “water soluble solvent” and “water-miscible solvent” and means a compound that is liquid at 25° C. and at atmospheric pressure (760 mmHg), and it has a solubility of at least 50% in water under these conditions. In some cases, the water-soluble solvents has a solubility of at least 60%, 70%, 80%, or 90%. Non-limiting examples of water-soluble solvents include, for example, organic solvents selected from glycerin, alcohols (for example C1-8, or C14 alcohols), polyols (polyhydric alcohols), glycols, and a mixture thereof.

Nonlimiting examples of water-soluble organic solvents. Non-limiting examples of water-soluble organic solvents include, for example, organic solvents selected from glycerin, alcohols (for example, C1-10, C1-8, or C1-4 alcohols), polyols (polyhydric alcohols), glycols, and a mixture thereof. Nonlimiting examples of monoalcohols and polyols include ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin.

Further non-limiting examples of water soluble organic solvents include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

In a preferred embodiment, the alkaline composition includes one or more glycols selected from glycerin, propylene glycol, butylene glycol, pentylene glycol, hexylene glycol, caprylyl glycol, dipropylene glycol, a C2-C6 monoalcohol (such as ethanol or isopropanol), and mixtures thereof.

The total amount of the one or more water-soluble solvents in the alkaline composition will vary depending on a variety of factors. Nonetheless, in various embodiments, the alkaline compositions include about 0.1 to about 20 wt. % of the one or more water soluble solvents, based on the total weight of the alkaline compositions. In further embodiments, the alkaline compositions include about 0.1 to about 15 wt. %, about 0.1 to about 12 wt. % about 0.1 to about 10 wt. %, about 0.5 to about 20 wt. %, about 0.5 to about 15 wt. %, about 0.5 to about 12 wt. %, about 0.5 to about 10 wt. %, about 1 to about 20 wt. %, about 1 to about 15 wt. %, about 1 to about 12 wt. %, about 1 to about 10 wt. %, about 2 to about 20 wt. %, about 2 to about 15 wt. %, about 2 to about 12 wt. %, about 2 to about 10 wt. %, about 5 to about 20 wt. %, about 5 to about 15 wt. %, about 5 to about 12 wt. %, or about 5 to about 10 wt. % of the one or more water-soluble solvents, based on a total weight of the alkaline composition.

### (I)(e) Nonionic Surfactants or Emulsifiers

The terms “nonionic surfactant” and “nonionic emulsifier” are used interchangeably in the instant disclosure and therefore can be referred to as “nonionic emulsifying surfactants.” The nonionic surfactant or emulsifier may have an HLB (hydrophilic-lipophilic balance) ranging from 1 to 7.9 or greater than or equal to 8. “HLB” refers to the “hydrophilic-lipophilic balance” associated with nonionic surfactants or emulsifiers. In particular, “HLB” value relates to the ratio of hydrophilic groups and lipophilic groups in emulsifiers, and also relates to solubility of the emulsifiers. Lower HLB emulsifiers (such as those with HLB values ranging from 1 to 7.9) are more soluble in oils (lipophilic material) and are more appropriate for use in water-in-oil (W/O) emulsions. Higher HLB emulsifiers (such as those with HLB values higher than 8) are more soluble in water (hydrophilic material) and are more appropriate for oil-in-water (O/W) emulsions.

Nonlimiting examples of nonionic surfactants or emulsifiers include alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, in particular alkyl and polyalkyl glucosides or polyglucosides, alkyl and polyalkyl esters of sucrose, optionally polyoxyethylenated alkyl and polyalkyl esters of glycerol, and optionally polyoxyethylenated alkyl and polyalkyl ethers of glycerol, and mixtures thereof. Preferably, the non-ionic surfactant(s) may be chosen from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, optionally polyoxyethylenated alkyl and polyalkyl esters of glycerol, and optionally polyoxyethylenated alkyl and polyalkyl ethers of glycerol, and mixtures thereof.
(1) Alkyl and polyalkyl esters of poly(ethylene oxide) that are preferably used are those containing at least one C8-C30 alkyl radical, with a number of ethylene oxide (EO) units ranging from 2 to 200. Mention may be made, for example, of (INCI name) PEG-20 stearate, PEG-40 stearate, PEG-100 stearate, PEG-20 laurate, PEG-8 laurate, PEG-40 laurate, PEG-150 distearate, PEG-7 cocoate, PEG-9 cococate, PEG-8 oleate, PEG-10 oleate and PEG-40 hydrogenated castor oil.
(2) Alkyl and polyalkyl ethers of poly(ethylene oxide) that are preferably used are those containing at least one C8-C30 alkyl radical, with a number of ethylene oxide (EO) units ranging from 3 to 200. Mention may be made, for example, of laureth-3, laureth-4, laureth-7, laureth-23, ceteth-5, ceteth-7, ceteth-15, ceteth-23, oleth-5, oleth-7, oleth-10, oleth-12, oleth-20, oleth-50, phytosterol 30 EO, steareth-6, steareth-20, steareth-21, steareth-40, steareth-100, beheneth 100, ceteareth-7, ceteareth-10, ceteareth-15, ceteareth-25, pareth-3, pareth-23, C12-15 pareth-3, C12-13 pareth-4, C12-13 pareth-23, trideceth-3, trideceth-4, trideceth-5, trideceth-6, trideceth-7 and trideceth-10, and mixtures thereof.
(3) Polyoxyethylenated alkyl and polyalkyl esters of sorbitan that are preferably used are those with a number of ethylene oxide (EO) units ranging from 0 to 100. Mention may be made, for example, of sorbitan laurate, sorbitan laurate 4 EO, sorbitan laurate 20 EO (polysorbate 20), sorbitan palmitate 20 EO (polysorbate 40), sorbitan stearate 20 EO (polysorbate 60), sorbitan oleate 20 EO (polysorbate 80) and sorbitan trioleate 20 EO (polysorbate 85).
(4) Polyoxyethylenated alkyl and polyalkyl ethers of sorbitan that are preferably used are those with a number of ethylene oxide (EO) units ranging from 0 to 100.

The compositions of the instant disclosure may include one or more alkanolamides. Non-limiting examples alkanolamides include fatty acid alkanolamides. The fatty acid alkanolamides may be fatty acid monoalkanolamides or fatty acid dialkanolamides or fatty acid isoalkanolamides, and may have a C₂₋₈ hydroxyalkyl group (the C₂₋₈ chain can be substituted with one or more than one —OH group). Non-limiting examples include fatty acid diethanolamides (DEA) or fatty acid monoethanolamides (MEA), fatty acid monoisopropanolamides (MIPA), fatty acid diisopropanolamides (DIPA), and fatty acid glucamides (acyl glucamides).

Suitable fatty acid alkanolamides include those formed by reacting an alkanolamine and a C6-C36 fatty acid. Examples include, but are not limited to: oleic acid diethanolamide, myristic acid monoethanolamide, soya fatty acids diethanolamide, stearic acid ethanolamide, oleic acid monoisopropanolamide, linoleic acid diethanolamide, stearic acid monoethanolamide (Stearamide MEA), behenic acid monoethanolamide, isostearic acid monoisopropanolamide (isostearamide MIPA), erucic acid diethanolamide, ricinoleic acid monoethanolamide, coconut fatty acid monoisopropanolamide (cocoamide MIPA), coconut acid monoethanolamide (Cocamide MEA), palm kernel fatty acid diethanolamide, coconut fatty acid diethanolamide, lauric diethanolamide, polyoxyethylene coconut fatty acid monoethanolamide, coconut fatty acid monoethanolamide, lauric monoethanolamide, lauric acid monoisopropanolamide (lauramide MIPA), myristic acid monoisopropanolamide (Myristamide MIPA), coconut fatty acid diisopropanolamide (cocamide DIPA), and mixtures thereof.

In some instances, the fatty acid alkanolamides preferably include cocamide MIPA, cocamide DEA, cocamide MEA, cocamide DIPA, and mixtures thereof. In particular, the fatty acid alkanolamide may be cocamide MIPA, which is commercially available under the tradename EMPILAN from Innospec Active Chemicals.

Fatty acid alkanolamides include those of the following structure:

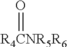

wherein R₄ is an alkyl chain of 4 to 20 carbon atoms (R₄ may be, for example, selected from lauric acid, coconut acid, palmitic acid, myristic acid, behenic acid, babassu fatty acid, isostearic acid, stearic acid, corn fatty acid, soy fatty acid, shea butter fatty acids, caprylic acid, capric acid, and mixtures thereof);
R₆ is selected from —CH₂OH, —CH₂CH₂OH, —CH₂CH₂CH₂OH, —CH₂(CHOH)₄CH₂OH, -benzyl, and mixtures thereof;
R₆ is selected from —H, —CH₃, —CH₂OH, —CH₂CH₃, —CH₂CH₂OH, —CH₂CH₂CH₂OH, —CH₂(CHOH)₄CH₂OH, -benzyl, and mixtures thereof.

In some instances, the one or more of the fatty acid alkanolamides include one or more acyl glucamides, for example, acyl glucamides having a carbon chain length of 8 to 20. Non-limiting examples include lauroyl/myristoyl methyl glucamide, capryloyl/capryl methyl glucamide, lauroyl methyl glucamide, myristoyl methyl glucamide, capryloyl methyl glucamide, capryl methyl glucamide, cocoyl methyl glucamide, capryloyl/caproyl methyl glucamide, cocoyl methyl glucamide, lauryl methylglucamide, oleoyl methylglucamide oleate, stearoyl methylglucamide stearate, sunfloweroyl methylglucamide, and tocopheryl succinate methylglucamide.

The compositions of the instant disclosure may include one or more alkyl polyglucosides. Non-limiting examples of alkyl polyglucosides include those having the following formula:

R¹—O—(R²O)ₙ—Z(x)
wherein R¹ is an alkyl group having 8-18 carbon atoms;
R² is an ethylene or propylene group;
Z is a saccharide group with 5 to 6 carbon atoms;
n is an integer from 0 to 10; and
x is an integer from 1 to 5.

Useful alkyl poly glucosides include lauryl glucoside, octyl glucoside, decyl glucoside, coco glucoside, caprylyl/capryl glucoside, and sodium lauryl glucose carboxylate. Typically, the at least one alkyl poly glucoside compound is selected from the group consisting of lauryl glucoside, decyl glucoside and coco glucoside. In some instances, decyl glucoside is particularly preferred.

The compositions of the instant disclosure may include one or more miscellaneous nonionic surfactants or emulsifiers. Nonlimiting examples include alcohols, alpha-diols, alkylphenols and esters of fatty acids, being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁₀-C₁₄)acylaminopropylmorpholine oxides; and mixtures thereof.

Such nonionic surfactants may preferably be chosen from polyoxyalkylenated or polyglycerolated nonionic surfactants. The oxyalkylene units are more particularly oxyethylene or oxypropylene units, or a combination thereof, and are preferably oxyethylene units.

In some cases, the nonionic surfactant may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and mixtures thereof.

Examples of ethoxylated fatty esters that may be mentioned include the adducts of ethylene oxide with esters of lauric acid, palmitic acid, stearic acid or behenic acid, and mixtures thereof, especially those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and mixtures thereof.

As glyceryl esters of fatty acids, glyceryl stearate (glyceryl mono-, di- and/or tristearate) (CTFA name: glyceryl stearate) or glyceryl ricinoleate and mixtures thereof can in particular be cited.

As glyceryl esters of C₈-C₂₄ alkoxylated fatty acids, polyethoxylated glyceryl stearate (glyceryl mono-, di- and/or tristearate) such as PEG-20 glyceryl stearate can for example be cited.

Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEG1N by Goldschmidt (CTFA name: glyceryl stearate SE), can also be used.

The total amount of the one or more nonionic surfactants or emulsifiers in the alklaine compositions will vary. Nonetheless, in various embodiments, the alkaline compositions include about 0.1 to about 10 wt. % of the one or more nonionic surfactants or emulsifiers. In further embodiments, the compositions include about 0.1 to about 8 wt. %, about 0.1 to about 5 wt. %, about 0.5 to about 10 wt. %, about 0.5 to about 8 wt. %, about 0.5 to about 5 wt. %, about 1 to about 10 wt. %, about 1 to about 8 wt. %, about 1 to about 5 wt. %, about 2 to about 10 wt. %, about 2 to about 8 wt. %, or about 2 to about 5 wt. % of the one or more nonionic surfactants or emulsifiers, based on a total weight of the alkaline composition.

### (I)(f) Polysaccharide Thickening Agents

The term “polysaccharides” refers to compounds containing a backbone of repeating sugar (i.e., carbohydrate) units. As the name suggests, a “polysaccharide thickening agent” increases the viscosity of the composition (thickens the composition). The polysaccharide thickening agents may be cationic, nonionic, or anionic. In various embodiments, the polysaccharide thickening agents are preferably nonionic polysaccharides.

Nonlimiting examples of polysaccharide thickening agents include starches, gums, celluloses, and mixtures thereof. Nonlimiting examples of starches include modified starches, starch-based polymers, methylhydroxypropyl starch, potato starch, wheat starch, rice starch, starch crosslinked with octenyl succinic anhydride (sold under the name Dry-Flo by National Starch), starch oxide, dialdehyde starch, dextrin, British gum, acetyl starch, starch phosphate, carboxymethyl starch, hydroxyethyl starch, and hydroxypropyl starch.

Nonlimiting examples of cellulose-based polymers include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename NATROSOL CS Plus from Aqualon Corporation.

In various embodiments, the polysaccharide thickening agents are preferably selected from scleroglucans comprising a linear chain of (1-3) linked glucose units with a (1-6) linked glucose every three units, a commercially available example of which is CLEAROGEL. CS1 1 from Michel Mercier Products Inc.

Nonlimiting examples of gums include acacia, agar, algin, alginic acid, ammonium alginate, amylopectin, calcium alginate, calcium carrageenan, carnitine, carrageenan, dextrin, gelatin, gellan gum, guar gum, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, biosacharide gum, and mixtures thereof.

In various embodiments, the one or more polysaccharide thickening agents are selected from hydroxypropyl starch phosphate, potato starch (modified or unmodified), wheat starch, rice starch, hydroxyethyl cellulose, guar gum, hydroxypropyl guar, xanthan gum, sclerotium gum, and a mixture thereof. In yet further embodiments, the polysaccharide is hydroxypropyl starch phosphate. Hydroxypropyl starch phosphate is sold under the tradename of STRUCTURE ZEA by the company Akzo Nobel. In a preferred embodiment, at least one of the one or more polysaccharide thickening agent comprises or consists of sclerotium gum, guar gum, a derivative thereof, or a combination thereof, for example, hydroxypropyl guar.

The total amount of the one or more polysaccharide thickening agents in the alkaline compositions will vary. Nonetheless, in various embodiments, the alkaline compositions include about 0.1 to about 8 wt. % of the one or more polysaccharides, based on the total weight of the composition. In further embodiments, the alkaline compositions include about 0.1 to about 6 wt. %, about 0.1 to about 5 wt. %, about 0.1 to about 4 wt. %, about 0.1 to about 3 wt. %, about 0.1 to about 2 wt. %, about 0.2 to about 8 wt. %, about 0.2 to about 6 wt. %, about 0.2 to about 5 wt. %, about 0.2 to about 4 wt. %, about 0.2 to about 3 wt. %, about 0.2 to about 2 wt. %, about 0.3 to about 8 wt. %, about 0.3 to about 6 wt. %, about 0.3 to about 5 wt. %, about 0.3 to about 5 wt. %, about 0.3 to about 4 wt. %, about 0.3 to about 3 wt. %, or about 0.3 to about 2 wt. % or the one or more polysaccharide thickening agents, based on the total weight of the composition.

### (I)(g) Silicones

In various embodiments, the leave-on hair treatment composition includes one or more silicones (also referred to as silicone-based oils). The one or more silicone-based oils can be volatile or non-volatile. Silicone-based oils include linear, branched, and cyclic silicone oils and include volatile and non-volatile silicone oils. Nonlimiting examples include polydimethylsiloxane (dimethicone), dimethiconol, amodimethicone, phenyl-modified silicone, silicone block copolymers containing amine groups or quat groups or other charged or uncharged silicone block copolymers, and blends of any of the foregoing. Additional nonlimiting examples include octamethylcyclotetrasiloxane, decamethylcyclopentasiloxane, dodecamethylcyclohexasiloxane, heptamethylhexyltrisiloxane, heptamethyl-octyltrisiloxane, hexamethyldisiloxane, octamethyltrisiloxane, decamethyltetrasiloxane and dodecamethylpentasiloxane, and mixtures thereof. In a preferred embodiment, alkaline composition includes at least one silicone-based oil selected from dimethicone, dimethiconol, amodimethicone, and a combination thereof, preferably dimethicone.

The total amount of the one or more silicones in the alkaline composition, if present, will vary. Nonetheless, in various embodiments the alkaline composition includes about 0.01 to about 10 wt. % of one or more silicones. In further embodiments, the alkaline composition includes about 0.01 to about 5 wt. %, about 0.01 to about 2 wt. %, about 0.01 to about 1 wt. %, about 0.05 to about 10 wt. %, about 0.05 to about 5 wt. %, about 0.05 to about 2 wt. %, about 0.05 to about 1 wt. % about 0.1 to about 10 wt. %, about 0.1 to about 5 wt. %, about 0.1 to about 2 wt. %, or about 0.1 to about 1 wt. % of the one or more silicones, based on a total weight of the alkaline composition.

### (I)(h) Miscellaneous Ingredients

The alkaline compositions optionally include or exclude (or are essentially free from) one or more miscellaneous ingredients. Miscellaneous ingredients are ingredients that are compatible with the compositions and do not disrupt or materially affect the basic and novel properties of the compositions. Nonlimiting examples of ingredients include preservatives (e.g., phenoxyethanol, benzoic acid, salicylic acid, etc.), fragrances, pH adjusters, salts, chelating agents, minerals (magnesium gluconate), buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, etc. In various embodiments, the miscellaneous ingredients are chosen from preservatives, fragrances, pH adjusters, salts, chelating agents, minerals, buffers, composition colorants, and mixtures thereof. In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair.

The total amount of the one or more miscellaneous ingredients in the alkaline composition, if present, will vary. Nonetheless, in various embodiments, the compositions include about 0.1 to about 15 wt. % of the one or more miscellaneous ingredients, based on the total weight of the compositions. In further embodiments, the compositions include about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. %, based on the total weight of the compositions.

The pH of the alkaline composition will vary but as the name suggest, the pH is alkaline (above 7). In various embodiments, the pH of the alkaline composition is about 9 to about 12. In further embodiments, the pH of the alkaline composition is about 9 to about 11, about 9 to about 10, about 10 to about 12, about 10 to about 11, about 11 to about 12, or about 9, about 9.5, about 10, about 10.5, about 11, about 11.5, or about 12.

### Embodiments of Alkaline Compositions

In various embodiments, the alkalizing composition of (I) comprises, consists essentially of, or consists of:
(a) about 1 to about 15 wt. %, preferably about 5 to about 15 wt. %, more preferably about 5 to about 12 wt. % of one or more alkalizing agents comprising:
   (a)(i) about 2 to 15 wt. %, preferably about 2 to about 12 wt. %, more preferably about 5 to about 10 wt. % of one or more alkanolamines, wherein the one or more alkanolamines are selected from ethanolamine, diethanolamine, triethanolamine, monopropanolamine, isopropanolamine, dipropanolamine, tripropanolamine, 2-amino-2-methyl-1,3-propanediol and 2-amino-2-hydroxymethyl-1,3-propanediol, and mixtures thereof, more preferably wherein the one or more alkanolamines comprises or consists of ethanolamine; and
   (a)(ii) optionally, about 1 to about 8 wt. %, preferably about 2 to about 5 wt. %, more preferably about 2 to about 6 wt. % of one or more additional alkalizing agents, preferably wherein at least one of the one or more additional alkalizing agents is selected from amino acids, hydroxide bases, or a combination thereof, more preferably wherein one or more amino acids are selected from aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine, ornithine, citrulline, and valine (preferably taurine) and the one or more hydroxide alkalizing agents are selected from ammonium hydroxide, sodium hydroxide, potassium hydroxide, lithium hydroxide, rubidium hydroxide, cesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminum hydroxide, guanidinium hydroxide and mixtures thereof (preferably sodium hydroxide); and
(b) about 50 to about 80 wt. %, preferably about 55 to about 75 wt. %, more preferably about 60 to about 70 wt. % of water;
(c) about 1 to about 20 wt. %, preferably about 2 to about 15 wt. %, more preferably about 5 to about 12 wt. % of one or more non-silicone based fatty compounds comprising:
   (c)(i) about 1 to about 12 wt. %, preferably about 2 to about 10 wt. %, more preferably about 2 to about 8 wt. % of one or more fatty alcohols, wherein the one or more fatty alcohols are preferably selected from decyl alcohol, undecyl alcohol, dodecyl, myristyl, cetyl alcohol, stearyl alcohol, cetearyl alcohol, isostearyl alcohol, isocetyl alcohol, behenyl alcohol, linalool, oleyl alcohol, myricyl alcohol and a mixture thereof, more preferably wherein the one or more fatty alcohols are selected cetyl alcohol, behenyl alcohol, cetearyl alcohol, or a combination thereof;
   (c)(ii) optionally, about 0.1 to about 18 wt. %, preferably about 1 to about 12 wt. %, more preferably about 3 to about 10 wt. % of one or more additional fatty compounds, preferably wherein the one or more additional fatty compounds are selected from C6-C16 hydrocarbons, being linear, branched or optionally cyclic, and are preferably alkanes, more preferably wherein the one or more additional fatty compounds are selected from hexane, dodecane and isoparaffins such as isohexadecane and isodecane, C13-C16 isoalkane or C13-C16 isoparaffin, or a combination thereof;
(d) about 0.1 to about 20 wt. %, preferably about 1 to about 15 wt. %, more preferably about 5 to about 15 wt. % of one or more water soluble solvents, preferably wherein the one or more water-soluble solvents are selected from glycerin, mono-alcohols (for example, C1-10, C1-8, or C1-4 alcohols), polyols (polyhydric alcohols), glycols, and a mixture thereof, more preferably wherein the one or more water soluble solvents are selected from one or more glycols selected from glycerin, propylene glycol, butylene glycol, pentylene glycol, hexylene glycol, caprylyl glycol, dipropylene glycol, a C2-C6 monoalcohol (such as ethanol or isopropanol), and mixtures thereof;
(e) about 0.1 to about 10 wt. %, preferably about 1 to about 10 wt. %, more preferably about 2 to about 8 wt. % of one or more nonionic surfactants or emulsifiers, preferably wherein at least one of the one or more nonionic surfactants or emulsifiers is/are selected from alkyl and polyalkyl ethers of poly(ethylene oxide), for example, containing at least one C9-C3-alkyl radical, with a number of ethylene oxide (EO units ranging from 2 to 200);
(f) about 0.1 to about 8 wt. %, preferably about 0.2 to about 6 wt. %, more preferably about 0.3 to about 3 wt. % of one or more polysaccharide thickening agents, preferably wherein the one or more polysaccharide thickening agents are selected from acacia, agar, algin, alginic acid, ammonium alginate, amylopectin, calcium alginate, calcium carrageenan, carnitine, carrageenan, dextrin, gelatin, gellan gum, guar gum, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, biosacharide gum, and mixtures thereof, more preferably wherein the one or more polysaccharide thickening agent comprises or consists of hydroxypropyl guar;
(g) optionally, about 0.01 to about 8 wt. %, preferably about 0.05 to about 5 wt. %, more preferably about 0.1 to about 3 wt. % one or more silicones, preferably wherein the one or more silicones are selected from polydimethylsiloxane (dimethicone), dimethiconol, amodimethicone, phenyl-modified silicone, silicone block copolymers containing amine groups or quat groups or other charged or uncharged silicone block copolymers, or a combination thereof, more preferably wherein the one or more silicones comprises or consists of dimethicone, dimethiconol, amodimethicone, or a combination thereof;
(h) optionally, about 0.01 to about 10 wt. %, preferably about 0.1 to about 8 wt. %, more preferably about 1 to about 5 wt. % of one or more miscellaneous ingredients, preferably selected from preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, silica, particular materials, etc.), emollients, composition colorants, or a mixture thereof;
   wherein all percentages by weight are based on a total weight of the composition.
      Application and Processing with Alkaline Composition

The alkaline composition can be applied to wet, damp, or dry hair. Nonetheless, the alkaline composition is typically applied to damp hair, for example, damp hair that has been washed shortly before treatment with the alkaline composition. For example, the hair may have been washed within 1 hour before application of the alkaline composition. Nonetheless, often the alkaline composition is applied more quickly after shampooing the hair, assuming the hair is cleansed/shampooed prior to application of the alkaline compositions. For example, the alkaline composition may be applied to the hair within 30 minutes, within 15 minutes, within 10 minutes, within 5 minutes from cleansing/shampooing the hair.

The alkaline composition is distributed throughout the hair and allowed to remain on the hair (process) for a period of time. Typically, the alkaline composition is allowed to remain on the hair for a period of time sufficient for the alkaline composition to open the hair cuticle and facilitate straightening. Nonlimiting example include about 1 minutes to about 60 minutes, about 1 minute to about 45 minutes, about 1 minute to about 30 minutes, about 1 minute to about 20 minutes, about 2 minutes to about 60 minutes, about 2 minute to about 45 minutes, about 2 minute to about 30 minutes, about 2 minute to about 20 minutes, about 3 minutes to about 60 minutes, about 3 minute to about 45 minutes, about 3 minute to about 30 minutes, about 3 minute to about 20 minutes, about 5 minutes to about 60 minutes, about 5 minute to about 45 minutes, about 5 minute to about 30 minutes, about 5 minute to about 20 minutes, or about 10 minutes to about 20 minutes.

After remaining on the hair for the period of time, the alkaline composition is typically rinsed from the hair with water. The hair may optionally be cleansed/shampooed after the alkaline composition has remained on the hair for the period of time. After the hair has been treated with the alkaline composition, it is subsequently treated with the conditioning composition, described in more detail below. Typically, the hair is treated with the conditioning compositions shortly after completion of the treatment with the alkaline composition, for example, within 1 hour after completion of the treatment with the alkaline composition. In various embodiments, the hair is treated with the conditioning composition within about 45 minutes, about 30 minutes, about 15 minutes, about 10 minutes, or about 5 minutes of completion of the treatment with the alkaline composition

### Conditioning Composition

### (II)(a) Citric Acid and Salts Thereof

The total amount of citric acid, salts thereof, or combination thereof in the conditioning compositions will vary. Nonetheless, in various embodiments, the conditioning composition includes about 1 to about 8 wt. % of citric acid, salts thereof, or a combination thereof, based on a total weight of the composition. In further embodiments, the conditioning composition includes about 1 to about 6 wt. %, about 1 to about 4 wt. %, about 1 to about 3 wt. %, about 2 to about 8 wt. %, about 2 to about 6 wt. % about 2 to about 5 wt. %, about 2 to about 4 wt. %, about 2 to about 3 wt. %, about 2.5 to about 8 wt. %, about 2.5 to about 6 wt. %, about 2.5 to about 5 wt. %, about 2.5 to about 4 wt. %, about 2.5 to about 3 wt. %, or about 1, about 1.5, about 2, about 2.5, about 3, about 3.5, about 4, about 4.5, or about 5 wt. % of citric acid, salts thereof, or a combination thereof, based on a total weight of the composition.

### (II)(b) Cyclodextrin and Derivatives

Compositions according to the disclosure comprise at least one cyclodextrin, a derivative thereof, or a combination thereof. Cyclodextrins are a family of cyclic oligosaccharides consisting of a macrocyclic ring of glucose subunits joined by α-1,4 glycosidic bonds.

The cyclodextrins that can be used include those of the formula:

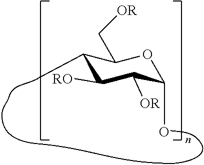

wherein:
R is chosen from H, CH₃, or a hydroxypropyl group, and
n ranges from 6-8.

For example, in embodiments where R=H, the cyclodextrin may be α-cyclodextrin (n=6), β-cyclodextrin (n=7), or γ-cyclodextrin (n=8). By way of example, α-cyclodextrin sold by the company WACKER under the name CAVAMAX W6 PHARMA, β-cyclodextrin sold by the company WACKER under the name CAVAMAX W7 PHARMA, or γ-cyclodextrin sold by the company WACKER under the name CAVAMAX W8 PHARMA can be used.

In other embodiments where R=CH₃, the cyclodextrin may be a methyl-cyclodextrin, such as methyl-α-cyclodextrin (n=6), methyl-β-cyclodextrin (n=7), or methyl-γ-cyclodextrin (n=8). For example, the methyl-β-cyclodextrin sold by the company WACKER under the name CAVASOL W7 may be chosen.

In various embodiments, the at least one cyclodextrin may comprise a mixture of cyclodextrins and/or derivatives thereof. For example, the at least one cyclodexctrin may be a mixture of α-cyclodextrin, β-cyclodextrin, and/or γ-cyclodextrin. In another embodiment, the at least one cyclodextrin includes β-cyclodextrin. In yet a further embodiment, the cyclodextrin is only β-cyclodextrin, and no other cyclodextrins or derivatives thereof are present in the composition.

In one embodiment, the compositions according to the present disclosure includes β-cyclodextrin in an amount ranging from about 0.1% to about 10%, such as from 0.2% to about 8%, from about 0.3% to about 7%, from about 0.4% to about 6%, from about 1% to about 10%, from about 1% to about 8%, from about 1% to about 5%, from about 1% to about 3% by weight, relative to the total weight of the hair treatment composition.

The total amount of cyclodextrin in the conditioning compositions will vary. Nonetheless, in various embodiments, the conditioning composition includes about 0.5 to about 5 wt. % of cyclodextrin, based on a total weight of the composition. In further embodiments, the conditioning composition includes 0.5 to about 4 wt. %, about 0.5 to about 3 wt. %, about 0.5 to about 2 wt. %, about 1 to about 5 wt. %, about 1 to about 4 wt. %, about 1 to about 3 wt. %, about 1 to about 2 wt. %, about 1.5 to about 5 wt. %, about 1.5 to about 4 wt. %, about 1.5 to about 3 wt. %, about 1.5 to about 2 wt. %, about 0.5 wt. %, about 1 wt. %, about 1.25 wt. %, about 1.5 wt. %, about 2 wt. %, about 2.5 wt. %, about 3 wt. %, about 3.5 wt. %, about 4 wt. %, about 4.5 wt. %, or about 5 wt. %, based on a total weight of the composition.

### Combination of Citric Acid/Salts and Cyclodextrin

The total combined amount of the citric acid, salts thereof, or combination of (a) and the cyclodextrin, derivatives thereof, or combination thereof of (b) will vary. Nonetheless, in various embodiments the total combined amount of the citric acid, salts thereof, or combination of (a) and the cyclodextrin, derivatives thereof, or combination thereof of (b) is about 1 to about 12 wt. %, based on a total weight of the hair treatment composition. In further embodiments, the total combined amount of the citric acid, salts thereof, or combination of (a) and the cyclodextrin, derivatives thereof, or combination thereof of (b) is about 1 to about 10 wt. %, about 1 to about 8 wt. %, about 1 to about 6 wt. %, about 1 to about 5 wt. %, about 2 to about 12 wt. %, about 2 to about 10 wt. %, about 2 to about 8 wt. %, about 2 to about 6 wt. %, about 2 to about 5 wt. %, about 3 to about 12 wt. %, about 3 to about 10 wt. %, about 3 to about 8 wt. %, about 3 to about 6 wt. %, about 3 to about 5 wt. %, or about 1 wt. %, 2 wt. %, 3 wt. %, 4 wt. %, 5 wt. %, 6 wt. %, 7 wt. %, or 8 wt. %, based on a total weight of the hair treatment composition.

The weight ratio of the citric acid, salts thereof, or combination of (a) to the cyclodextrin, derivatives thereof, or combination thereof of (b) will vary. Nonetheless, in various embodiments, the citric acid, salts thereof, or combination of (a) and the cyclodextrin, derivatives thereof, or combination thereof of (b) are in a weight ratio of about of about 8:1 to about 1:2((a):(b)). In further embodiments, the citric acid, salts thereof, or combination of (a) and the cyclodextrin or derivatives of (b) are in a weight ratio of 6:1 to about 1:2, about 5:2 to about 1:2, about 4:1 to about 1:2, about 3:1 to about 1:2, about 2:1 to about 1:2, about 8:1 to about 1:1, about 6:1 to about 1:1, about 5:1 to about 1:1, about 4:1 to about 1:1, about 3:1 to about 1:1, about 2:1 to about 1:1, about 1.2:1, about 1.3:1, about 1.4:1, about 1.5:1, about 1.6:1, or about 1.8:1 ((a):(b)).

The mole ratio of the citric acid, salts thereof, or combination of (a) to the cyclodextrin, derivatives thereof, or combination thereof of (b) will vary. Nonetheless, in various embodiments, the citric acid, salts thereof, or combination of (a) and the cyclodextrin, derivatives thereof, or combination thereof of (b) are in a mole ratio of about 20:1 to about 3:1. In further embodiments, the citric acid, salts thereof, or combination of (a) and the cyclodextrin or derivatives of (b) are in a mole ratio of about 18:1 to about 3:1, about 15:1 to about 3:1, about 20:1 to about 5:1, about 18:1 to about 5:1, about 15:1 to about 5:1, about 20:1 to about 8:1, about 18:1 to about 8:1, about 15:1 to about 8:1, about 20:1 to about 10:1, about 18:1 to about 10:1, about 15:1 to about 10:1, about 14:1, about 13:1, about 12:1, or about 11:1.

In various embodiments, the citric acid, salts thereof, or combination of (a) and the cyclodextrin, derivatives thereof, or combination thereof of (b) are combined with one another before being added into the hair treatment compositions of the instant disclosure. For example, the cyclodextrin, derivatives thereof, or combination thereof is preferably solubilized in the citric acid, salt thereof, or combination thereof to form a solubilized combination of citric acid, salt thereof, or combination thereof and the cyclodextrin, derivatives thereof, or combination thereof. The combination can be heated to facilitate or hasten the dissolution of the cyclodextrin and/or derivatives thereof. The solubility of cyclodextrin and/or derivative thereof in water is not always ideal. Therefore, combining the cyclodextrin and/or derivative thereof with the citric acid, salt thereof, or combination thereof, and dissolving the cyclodextrin and/or derivative thereof in citric acid, salt thereof, or combination before adding the combination to other components of the conditioning composition can be beneficial.

### (II)(c) Cationic Surfactants

The term “cationic surfactant” as defined by the instant disclosure is a surfactant that may be positively charged when it is contained in the hair treatment compositions according to the disclosure. The cationic surfactant may bear one or more positive permanent charges or may contain one or more functional groups that are cationizable in the composition according to the disclosure.

Mono-alkyl cationic surfactants useful herein are primary, secondary, and tertiary amines having one long alkyl or alkenyl group of from about 12 to about 30 carbon atoms, preferably from 16 to 24 carbon atoms, more preferably from 18 to 22 alkyl group. For example, mono-alkyl cationic surfactants include mono-alkyl trimonium halide compounds. Nonlimiting examples of mono-alkyl trimonium halide compounds include cetrimonium chloride, steartrimonium chloride, behentrimonium chloride, cocotrimonium chloride, cocamidopropyltrimonium chloride. Preferred are cetrimonium chloride, steartrimonium chloride and behentrimonium chloride.

In various embodiments, the hair treatment compositions includes behentrimonium chloride, cetrimonium chloride, or a combination thereof.

Mono-alkyl cationic surfactants also include mono-alkyl amidoamines. Particularly useful are tertiary amidoamines having an alkyl group of from about 12 to about 22 carbon atoms, preferably from about 16 to about 22 carbon atoms. Exemplary tertiary amido amines include: stearamidopropyldimethylamine, stearamidopropyl-diethylamine, stearamidoethyldiethylamine, stearamidoethyldimethylamine, palmitamidopropyldimethylamine, palmitamidopropyldiethylamine, palmitamidoethyl-diethylamine, palmitamidoethyldimethylamine, behenamidopropyldimethylamine, behenamidopropyldiethylamine, behenamidoethyldiethylamine, behenamidoethyl-dimethylamine, arachidamidopropyldimethylamine, arachidamidopropyldiethylamine, arachidamidoethyldiethylamine, arachidamidoethyldimethylamine, diethylaminoethyl-stearamide, and a combination thereof.

Di-alkyl cationic surfactants includes those of formula (I) and salts thereof:

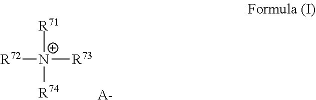

wherein two of R⁷¹, R⁷², R⁷³, and R⁷⁴ are selected from an aliphatic group of from 12 to 30 carbon atoms, preferably from 16 to 24 carbon atoms, more preferably from 18 to 22 carbon atoms or an aromatic, alkoxy, polyoxyalkylene, alkylamido, hydroxyalkyl, aryl or alkylaryl group having up to about 30 carbon atoms;
the remainder of R⁷¹, R⁷², R⁷³ and R⁷⁴ are independently selected from an aliphatic group of from 1 to about 8 carbon atoms, preferably from 1 to 3 carbon atoms or an aromatic, alkoxy, polyoxyalkylene, alkylamido, hydroxyalkyl, aryl or alkylaryl group having up to about 8 carbon atoms; and
A⁻ is an anion, for example, a halide, such as chloride or bromide, a C1-C4 alkyl sulfate such as methosulfate and ethosulfate, and mixtures thereof.

The aliphatic groups for Formula (I) can contain, in addition to carbon and hydrogen atoms, ether linkages, and other groups such as amino groups. The longer chain aliphatic groups, e.g., those of about 16 carbons, or higher, can be saturated or unsaturated. Preferably, two of R⁷¹, R⁷², R⁷³ and R⁷⁴ are selected from an alkyl group of from 12 to 30 carbon atoms, preferably from 16 to 24 carbon atoms, more preferably from 18 to 22 carbon atoms; and the remainder of R⁷¹, R⁷², R⁷³ and R⁷⁴ are independently selected from CH₃, C₂H₅, C₂H₄OH, CH₂C₆H₅, and mixtures thereof.

Nonlimiting examples of di-alkyl cationic surfactants of Formula (I) include dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, dicetyldimonium chloride, dicetyldimonium bromide, and a combination thereof.

In various embodiments, the one or more di-alkyl cationic surfactants are selected from di-alkyl dimonium halide compounds. Nonlimiting examples include dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, dicetyldimonium chloride, dicetyldimonium bromide, and a combination thereof. In a preferred embodiment, the di-alkyl dimonium halide compounds are selected from dicetyldimonium chloride, dicetyldimonium bromide, and a combination thereof. In a preferred embodiment, the hair treatment composition includes dicetyldimonium chloride.

The total amount of the one or more cationic surfactants in the hair treatment composition will vary. Nonetheless, in various embodiments, the hair treatment compositions include about 1 to about 10 wt. % of the one or more cationic surfactants, based on a total weight of the composition. In further embodiments, the hair treatment composition includes about 1 to about 8 wt. %, about 1 to about 6 wt. %, about 1 to about 5 wt. %, about 1 to about 4 wt. %, about 1 to about 3 wt. %, about 1.5 to about 10 wt. %, 1.5 to about 8 wt. %, about 1.5 to about 6 wt. %, about 1.5 to about 5 wt. %, about 1.5 to about 4 wt. %, about 1.5 to about 3 wt. %, about 2 to about 10 wt. %, about 2 to about 8 wt. %, about 2 to about 6 wt. %, about 2 to about 5 wt. %, about 2 to about 4 wt. %, about 2 to about 3 wt. %, or about 1 wt. %, 1.5 wt. %, 2 wt. %, about 2.5 wt. %, about 3 wt. %, about 3.5 wt. %, about 4 wt. %, about 4.5 wt. %, about 5 wt. %, about 6 wt. %, about 8 wt. %, or about 10 wt. % of the one or more cationic surfactants.

### (II)(d) Nonionic Surfactants or Emulsifiers

A nonlimiting but somewhat exhaustive list of useful nonionic surfactants and emulsifiers is set forth above, under the heading “(I)(e) Nonionic Surfactants or Emulsifiers” for the alkaline compositions, which is incorporated herein by reference. In other words, the nonionic surfactants or emulsifiers set forth in Section “(I)(e) Nonionic Surfactants or Emulsifiers” can be incorporated into the conditioning compositions. They are not reiterated here for the sake of brevity.

The total amount of the one or more nonionic surfactants or emulsifiers in the hair treatment compositions will vary. Nonetheless, in various embodiments, the hair treatment compositions include about 0.1 to about 10 wt. % of the one or more nonionic surfactants or emulsifiers. In further embodiments, the compositions include about 0.1 to about 8 wt. %, about 0.1 to about 5 wt. %, about 0.1 to about 3 wt. %, about 0.5 to about 10 wt. %, about 0.5 to about 8 wt. %, about 0.5 to about 5 wt. %, about 0.5 to about 3 wt. %, about 1 to about 10 wt. %, about 1 to about 8 wt. %, about 1 to about 5 wt. %, about 1 to about 3 wt. %, about 0.1 wt. %, about 0.5 wt. %, about 1 wt. %, about 1.5 wt. %, about 2 wt. %, about 2.5 wt. %, about 3 wt. % about 5 wt. %, about 6 wt. %, about 8 wt. %, or about 10 wt. % of the one or more nonionic surfactants or emulsifiers, based on a total weight of the compositions.

### (II)(e) Fatty Alcohols

A nonlimiting description and list of useful fatty alcohols is set forth above, under the heading “(l)(c)(i) Fatty Alcohols” for the alkaline compositions, which is incorporated herein by reference. In other words, the fatty alcohols set forth in Section “(l)(c)(i) Fatty Alcohols” can be incorporated into the conditioning compositions. They are not reiterated here for the sake of brevity. Nonetheless, in various embodiments, the conditioning compositions include one or more fatty alcohols selected from decyl alcohol, undecyl alcohol, dodecyl, myristyl, cetyl alcohol, stearyl alcohol, cetearyl alcohol, isostearyl alcohol, isocetyl alcohol, behenyl alcohol, linalool, oleyl alcohol, myricyl alcohol and a mixture thereof. In some instances, the cosmetic compositions preferably include cetyl alcohol, behenyl alcohol, cetearyl alcohol, or a combination thereof.

The total amount of the one or more fatty alcohols in the conditioning composition will vary. Nonetheless, in various embodiments, the hair treatment composition includes about 1 to about 15 wt. % of the one or more fatty alcohols, based on a total weight of the composition. In further embodiments, the hair treatment composition includes about 2 to about 15 wt. %, about 3 to about 15 wt. %, about 4 to about 15 wt. %, about 5 to about 15 wt. %, about 6 to about 15 wt. %, about 1 to about 12 wt. %, about 2 to about 12 wt. %, about 3 to about 12 wt. %, about 4 to about 12 wt. %, about 5 to about 12 wt. %, about 6 to about 12 wt. %, about 1 to about 10 wt. %, about 2 to about 10 wt. %, about 3 to about 10 wt. %, about 4 to about 10 wt. %, about 5 to about 10 wt. %, about 6 to about 10 wt. %, about 1 to about 8 wt. %, about 2 to about 8 wt. %, about 3 to about 8 wt. %, about 4 to about 8 wt. %, about 5 to about 10 wt. %, about 6 to 8 wt. %, about 2 wt. %, about 3 wt. %, about 4 wt. %, about 5 wt. %, about 6 wt. %, about 7 wt. %, or about 8 wt. % of the one or more fatty alcohols.

### (II)(f) Amino-Functionalized Silicones

The term “amino-functionalized silicone” or “amino silicones” means a silicone containing at least one primary amino, secondary amino, tertiary amino and/or quaternary ammonium group. The structure of the amino-functionalized silicone may be linear or branched, cyclic or non-cyclic. The amino functional group may be at any position in the silicone molecule, preferably at the end of the backbone (for example, in the case of amodimethicones) and/or in the side chain.

In some instances, an amino-functionalized silicones is selected from compounds having the following formula:

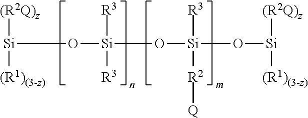

wherein each R¹ is independently selected from a C₁₋₃₀ alkyl group, a C₁₋₃₀ alkoxy group, a C₅₋₃₀ aryl group, a C₆₋₃₀ aralkyl group, a C₆₋₃₀ aralkyloxy group, a C₁₋₃₀ alkaryl group, a C₁₋₃₀ alkoxyaryl group, and a hydroxy group (preferably, each R¹ is independently selected from a C₁₋₃₀ alkyl group, a C₁₋₃₀ alkoxy group and a hydroxy group);
each R² is independently a divalent alkylene radical having one to ten carbon atoms (preferably, R² is a divalent alkylene radical having three to six carbon atoms); each R³ is independently selected from a C₁₋₃₀ alkyl group, a C₅₋₃₀ aryl group, a C₆₋₃₀ aralkyl group and a C₁₋₃₀ alkaryl group (preferably, each R³ is independently selected from of a C₁₋₃₀ alkyl group);
Q is a monovalent radical selected from —NR⁴₂ and —NR⁴(CH₂)ₓNR⁴₂;
each R⁴ is independently selected from a hydrogen and a C₁₋₄ alkyl group;
x is 2 to 6;
z is 0 or 1;
n is 25 to 3,000 (preferably, 25 to 2,000; more preferably, 25 to 1,000; most preferably 25 to 500); and
m is 0 to 3,000 (preferably, 0 to 2,000; more preferably, 0 to 1,000; most preferably, 0 to 100);
with the proviso that at least 50 mol % of the total number of R¹ and R³ groups are methyl and with the proviso that when m is 0, z is 1.

Preferred R¹ groups include methyl, methoxy, ethyl, ethoxy, propyl, propoxy, isopropyl, isopropoxy, butyl, butoxy, isobutyl, isobutoxy, phenyl, xenyl, benzyl, phenylethyl, tolyl and hydoxy. Preferred R² divalent alkylene radicals include trimethylene, tetramethylene, pentamethylene, —CH₂CH(CH₃)CH₂ and CH₂CH₂CH(CH₃)CH₂.

Preferred R³ groups include methyl, ethyl, propyl, isopropyl, butyl, isobutyl, phenyl, xenyl, benzyl, phenylethyl and tolyl. Preferred R⁴ groups include methyl, ethyl, propyl, isopropyl, butyl and isobutyl. When z is 0, the amino-functionalized silicine has only pendant amine functional substituents in the polymer chain. When z is 1, the amino-functional silicone may have only terminal amine functional substituents (e.g., m=0) or may have both terminal and pendant amine functional substituents in the polymer chain (e.g., m>0). Preferably, n+m is 50 to 1,000. More preferably, n+m is 50 to 750. Still more preferably, n+m is 50 to 500. Most preferably, n+m is 50 to 250.

In some instances, the amino-functionalized silicones are alkoxylated and/or hydroxylated amino silicones.

Non-limiting examples of amino-functionalized silicones include bis-hydroxy/methoxy amodimethicones, bis-cetearyl amodimethicone, amodimethicone, bis(C13-15 alkoxy) PG amodimethicones, aminopropyl phenyl trimethicones, aminopropyl dimethicones, bis-amino PEG/PPG-41/3 aminoethyl PG-propyl dimethicones, caprylyl methicones, and a mixture thereof. In some instances, a particularly useful amino-functionalized silicone is bis-hydroxy/methoxy amodimethicone, wherein X is isobutyl and one of the R is OH and the other is OCH₃ in the above structure, also known as “Bis-Hydroxy/Methoxy Amodimethicone” and “3-[(2-aminoethyl)amino]-2-methylpropyl Me, di-Me, [(hydroxydimethylsilyl)oxy]- and [(methoxydimethylsilyl)oxy]-terminated.” Bis-hydroxy/methoxy amodimethicone is commercially available under the tradename DOWSIL AP-8087 FLUID from The Dow Chemical Company. A particularly preferred amino-functionalized silicone is amodimethicone” A non-limiting example of amodimethicone products containing amino silicones having structure (D) re sold by Wacker under the name BELSIL ADM 652, BELSIL ADM 4000 E, or BELSIL ADM LOG 1. A product containing amino silicones having structure (E) is sold by Wacker under the name FLUID WR 1300. Additionally or alternative, the weight-average molecular weight (Mw) of the silicone ranges preferably from 2,000 to 200,000, even more particularly 5,000 to 100,000 and more particularly from 10,000 to 50,000.

In a preferred embodiment, the one or more amino-functionalized silicones are selected from amodimethicone, bis-hydroxy/methoxy amodimethicone, bis-cetearyl amodimethicone, bis(C13-15 alkoxy) PG amodimethicone, aminopropyl phenyl trimethicone, aminopropyl dimethicone, bis-amino PEG/PPG-41/3 aminoethyl PG-propyl dimethicone, or a mixture thereof. In a further preferred embodiments, the amino-functionalized silicone is amodimethicone.

The total amount of the one or more amino-functionalized silicones in the conditioning compositions will vary. Nonetheless, in various embodiments, the conditioning compositions include about 1 to about 18 wt. % of the one or more amino-functionalized silicones, based on the total weight of the composition. In further embodiments, the conditioning compositions include about 1 to about 15 wt. %, about 1 to 12 wt. %, about 1 to about 10 wt. %, about 1 to about 8 wt. %, about 1 to about 6 wt. %, about 2 to about 18 wt. %, about 2 to about 12 wt. %, about 2 to about 10 wt. %, about 2 to about 10 wt. %, about 2 to about 8 wt. %, about 2 to about 6 wt. %, about 3 to about 18 wt. %, about 3 to about 15 wt. %, about 3 to about 12 wt. %, about 3 to about 10 wt. %, about 3 to about 8 wt. %, about 3 to about 6 wt. %, about 4 to about 18 wt. %, about 4 to about 15 wt. %, about 4 to about 12 wt. %, about 4 to about 10 wt. %, about 4 to about 8 wt. %, about 4 to about 6 wt. %, about 1 wt. %, about 2 wt. %, about 3 wt. %, about 4 wt. %, about 5 wt. %, about 6 wt. %, about 7 wt. %, or about 8 wt. %, of the one or more amino-functionalized silicones based on the total weight of the composition.

### (II)(g) Water-Soluble Solvents

A nonlimiting list of useful water-soluble solvents is described above, under the heading “(I)(d) Water Soluble Solvents” for the alkaline compositions, which is incorporated herein by reference. In other words, the water-soluble solvents set forth in Section “(I)(d) Water Soluble Solvents” are useful in the conditioning compositions. They are not reiterated here for the sake of brevity.

The total amount of the one or more water-soluble solvents in the conditioning compositions, if present, will vary. Nonetheless, in various embodiments, the conditioning compositions include about 0.1 to about 20 wt. % of the one or more water soluble solvents, based on the total weight of the compositions. In further embodiments, the conditioning compositions include about 0.1 to about 15 wt. %, about 0.1 to about 10 wt. %, about 0.1 to about 8 wt. %, about 0.1 to about 5 wt. %, about 0.5 to about 20 wt. %, about 0.5 to about 15 wt. %, about 0.5 to about 10 wt. %, about 0.5 to about 8 wt. %, about 0.5 to about 5 wt. %, about 1 to about 20 wt. %, about 1 to about 15 wt. %, about 1 to about 10 wt. %, about 1 to about 8 wt. %, about 1 to about 5 wt. %, about 2 to about 20 wt. %, about 2 to about 15 wt. %, about 2 to about 10 wt. %, 2 to about 8 wt. %, about 2 to about 5 wt. %, about 1 wt. %, about 1.5 wt. %, about 2 wt. %, about 2.5 wt. %, about 3 wt. %, about 3.5 wt. %, about 4 wt. %, about 4.5 wt. %, or about 5 wt. % of the one or more water-soluble solvents, based on a total weight of the composition.

### (II)(h) Water

The total amount of water in the hair treatment compositions will vary. Nonetheless, in various embodiments, the hair treatment compositions include about 50 to about 90 wt. % of water, based on the total weight of the compositions. In further embodiments, the hair treatment composition includes about 60 to about 90 wt. %, about 70 to about 90 wt. %, about 50 to about 85 wt. %, about 60 to about 85 wt. %, about 70 to about 85 wt. %, about 50 to about 80 wt. %, about 60 to about 80 wt. %, about 70 to about 80 wt. %, about 65 to about 85 wt. %, about 60 wt. %, about 65 wt. %, about 70 wt. %, about 75 wt. %, about 80 wt. %, or about 85 wt. %, based on the total weight of the compositions.

### (II)(i) Non-Silicone Based Fatty Compounds

A nonlimiting list of useful non-silicone based fatty compounds is described above, under the heading “(I)(c) Non-Silicone Based Fatty Compound” for the alkaline compositions, which is incorporated herein by reference. In other words, the non-silicone based fatty compounds set forth in Section “(I)(c) Non-Silicone Based Fatty Compounds” are useful in the conditioning compositions. They are not reiterated here for the sake of brevity.

The total amount of the one or more non-silicone-based fatty compounds in the conditioning compositions will vary. Nonetheless, in various embodiments, the conditioning composition include about 0.1 to about 20 wt. % of the one or more non-silicone-based fatty compounds, based on the total weight of the compositions. In further embodiments, the compositions include about 0.1 to about 15 wt. %, about 0.1 to about 12 wt. %, about 0.1 to about 10 wt. %, about 0.1 to about 8 wt. %, about 0.1 to about 5 wt. %, about 0.5 to about 20 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 20 wt. %, about 1 to about 15 wt. %, about 1 to about 12 wt. %, about 1 to about 10 wt. %, about 1 to about 8 wt. %, about 1 to about 5 wt. %, about 2 to about 20 wt. %, about 2 to about 15 wt. %, about 2 to about 12 wt. %, about 2 to about 10 wt. %, about 2 to about 8 wt. %, about 2 to about 5 wt. %, or about 3 to about 5 wt. % of the one or more non-silicone based fatty compounds, based on the total weight of the conditioning compositions.

### (II)(j) Cationic Conditioning Polymer

Cationic polymers for purposes of the instant disclosure are polymers bearing a positive charge or incorporating cationic entities in their structure. The cationic polymers can comprise mixtures of monomer units derived from amine- and/or quaternary ammonium-substituted monomer and/or compatible spacer monomers. Cationic polymers often provide conditioning benefits to the hair treatment compositions and therefore may be referred to as “cationic conditioning polymers.” Non-limiting examples of cationic polymers include copolymers of 1-vinyl-2-pyrrolidine and 1-vinyl-3-methyl-imidazolium salt (e.g., chloride salt) (referred to as Polyquaternium-16); copolymers of 1-vinyl-2-pyrrolidine and dimethylaminoethyl methacrylate (referred to as Polyquaternium-11); cationic diallyl quaternary ammonium-containing polymer including, for example, dimethyldiallyammonium chloride homopolymer and copolymers of acrylamide and dimethyldiallyammonium chloride (referred to as Polyquaternium-6 and Polyquaternium-7); polysaccharide polymers, such as cationic cellulose derivatives and cationic starch derivatives. Cationic cellulose is available as salts of hydroxyethyl cellulose reacted with trimethyl ammonium substituted epoxide (referred to as Polyquaternium-10). Another type of cationic cellulose includes the polymeric quaternary ammonium salts of hydroxyethyl cellulose reacted with lauryl dimethyl ammonium-substituted epoxide (referred to as Polyquaternium-24). Additionally or alternatively, the cationic conditioning polymers may include or be chosen from cationic guar gum derivatives, such as guar hydroxypropyltrimonium chloride.

Preferred cationic polymers include cationic polysaccharide polymers, such as cationic cellulose, cationic starch, and cationic guar gum. In the context of the instant disclosure cationic polysaccharide polymers include cationic polysaccharides and polysaccharide derivatives (e.g., derivatized to be cationic), for example, resulting in cationic cellulose (cellulose derivatized to be cationic), cationic starch (derivatized to be cationic), cationic guar (guar derivatized to be cationic).

Non-limiting examples of cationic celluloses include hydroxyethylcellulose (also known as HEC), hydroxymethylcellulose, methylhydroxyethylcellulose, hydroxypropylcellulose (also known as HPC), hydroxybutylcellulose, hydroxyethylmethylcellulose (also known as methyl hydroxyethylcellulose) and hydroxypropylmethylcellulose (also known as HPMC), cetyl hydroxyethylcellulose, polyquaternium-10, polyquaternium-24, and mixtures thereof, preferably polyquaternium-10, polyquaternium-24, and mixtures thereof.

Non-limiting examples of cationic guar include guar hydroxypropyltrimonium chloride, hydroxypropyl guar hydroxypropyltrimonium chloride, guar hydroxypropyltrimethylammonium chloride, and mixtures thereof.

Non-limiting examples of cationic starch include starch hydroxypropyltrimonium chloride, hydroxypropyl oxidized starch PG trimonium chloride, and a mixture thereof.

In certain embodiments, the hair treatment composition may include one or more polyquaterniums. Nonlimiting examples include polyquaternium-1, polyquaternium-2, polyquaternium-3, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-21, polyquaternium-22, polyquaternium-23, polyquaternium-24, polyquaternium-25, polyquaternium-26, polyquaternium-27, polyquaternium-28, polyquaternium-29, polyquaternium-30, polyquaternium-40, polyquaternium-41, polyquaternium-42, polyquaternium-43, polyquaternium-44, polyquaternium-45, polyquaternium-46, polyquaternium-47, polyquaternium-48, polyquaternium-49, polyquaternium-50, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-61, polyquaternium-62, polyquaternium-63, polyquaternium-64, polyquaternium-65, polyquaternium-66, polyquaternium-67, etc. In some cases, preferred polyquaternium compounds include polyquaternium-10, polyquaternium-11, polyquaternium-67, and a mixture thereof.

In certain embodiments, the hair treatment composition may include polyquaternium-1 (ethanol, 2,2′,2″-nitrilotris-, polymer with 1,4-dichloro-2-butene and N,N,N′,N′-tetramethyl-2-butene-1,4-diamine), polyquaternium-2, (poly[bis(2-chloroethyl) ether-alt-1,3-bis[3-(dimethylamino)propyl]urea]), polyquaternium-4, (hydroxyethyl cellulose dimethyl diallylammonium chloride copolymer; Diallyldimethylammonium chloride-hydroxyethyl cellulose copolymer), polyquaternium-5 (copolymer of acrylamide and quaternized dimethylammoniumethyl methacrylate), polyquaternium-6 (poly(diallyldimethylammonium chloride)), polyquaternium-7 (copolymer of acrylamide and diallyldimethylammonium chloride), polyquaternium-8 (copolymer of methyl and stearyl dimethylaminoethyl ester of methacrylic acid, quaternized with dimethylsulphate), polyquaternium-9 (homopolymer of N,N-(dimethylamino)ethyl ester of methacrylic acid, quaternized with bromomethane), polyquaternium-10 (quaternized hydroxyethyl cellulose), polyquaternium-11 (copolymer of vinylpyrrolidone and quaternized dimethylaminoethyl methacrylate), polyquaternium-12 (ethyl methacrylate/abietyl methacrylate/diethylaminoethyl methacrylate copolymer quaternized with dimethyl sulfate), polyquaternium-13 (ethyl methacrylate/oleyl methacrylate/diethylaminoethyl methacrylate copolymer quaternized with dimethyl sulfate), polyquaternium-14 (trimethylaminoethylmethacrylate homopolymer), polyquaternium-15 (acrylamide-dimethylaminoethyl methacrylate methyl chloride copolymer), Polyquaternium-16 (copolymer of vinylpyrrolidone and quaternized vinylimidazole), Polyquaternium-17 (adipic acid, dimethylaminopropylamine and dichloroethylether copolymer), Polyquaternium-18 (azelanic acid, dimethylaminopropylamine and dichloroethylether copolymer), polyquaternium-19 (copolymer of polyvinyl alcohol and 2,3-epoxypropylamine), polyquaternium-20 (copolymer of polyvinyl octadecyl ether and 2,3-epoxypropylamine), polyquaternium-22 (copolymer of acrylic acid and diallyldimethylammonium chloride), polyquaternium-24 (auaternary ammonium salt of hydroxyethyl cellulose reacted with a lauryl dimethyl ammonium substituted epoxide), polyquaternium-27 (block copolymer of Polyquaternium-2 and Polyquaternium-17), polyquaternium-28 (copolymer of vinylpyrrolidone and methacrylamidopropyl trimethylammonium), polyquaternium-29 (chitosan modified with propylen oxide and quaternized with epichlorhydrin), polyquaternium-30 (ethanaminium, N-(carboxymethyl)-N,N-dimethyl-2-[(2-methyl-1-oxo-2-propen-1-yl)oxy]-, inner salt, polymer with methyl 2-methyl-2-propenoate), polyquaternium-31 (N,N-dimethylaminopropyl-N-acrylamidine quatemized with diethylsulfate bound to a block of polyacrylonitrile), polyquaternium-32 (poly(acrylamide 2-methacryloxyethyltrimethyl ammonium chloride)), polyquaternium-33 (copolymer of trimethylaminoethylacrylate salt and acrylamide), polyquaternium-34 (copolymer of 1,3-dibromopropane and N,N-diethyl-N′,N′-dimethyl-1,3-propanediamine), Polyquaternium-35 (methosulphate of the copolymer of methacryloyloxyethyltrimethylammonium and of methacryloyloxyethyldimethylacetylammonium), polyquaternium-36 (copolymer of N,N-dimethylaminoethylmethacrylate and buthylmethacrylate, quaternized with dimethylsulphate), polyquaternium-37 (poly(2-methacryloxyethyltrimethylammonium chloride)), polyquaternium-39 (terpolymer of acrylic acid, acrylamide and diallyldimethylammonium Chloride), polyquaternium-42 (poly[oxyethylene(dimethylimino)ethylene (dimethylimino)ethylene dichloride]), Polyquaternium-43 (copolymer of acrylamide, acrylamidopropyltrimonium chloride, 2-amidopropylacrylamide sulfonate and dimethylaminopropylamine), polyquaternium-44 (3-Methyl-1-vinylimidazolium methyl sulfate-N-vinylpyrrolidone copolymer), polyquaternium-45 (copolymer of (N-methyl-N-ethoxyglycine)methacrylate and N,N-dimethylaminoethylmethacrylate, quaternized with dimethyl sulphate), polyquaternium-46 (terpolymer of vinylcaprolactam, vinylpyrrolidone, and quaternized vinylimidazole), polyquaternium-47 (terpolymer of acrylic acid, methacrylamidopropyl trimethylammonium chloride, and methyl acrylate), and/or polyquaternium-67.

In certain embodiments, the hair treatment compositions of the instant disclosure include one or more cationic polymers selected from cationic cellulose derivatives, quaternized hydroxyethyl cellulose (e.g., polyquaternium-10), cationic starch derivatives, cationic guar gum derivatives, copolymers of acrylamide and dimethyldiallyammonium chloride (e.g., polyquaternium-7), polyquaterniums, and a mixture thereof. For example, the cationic polymer(s) may be selected from polyquaterniums, for example, polyquaterniums selected from polyquaternium-4, polyquaternium-5, polyquaternium-6, polyquaternium-7, polyquaternium-10, polyquaternium-22, polyquaternium-37, polyquaternium-39, polyquaternium-47, polyquaternium-53, polyquaternium-67 and a mixture thereof. A combination of two or more polyquaterniums can be useful. A particularly preferred and useful cationic polymer is polyquaternium-10.

In certain embodiments, the hair treatment compositions include one or more cationic polymers chosen from cationic proteins and cationic protein hydrolysates (e.g., hydroxypropyltrimonium hydrolyzed wheat protein), quaternary diammonium polymers (e.g., hexadimethrine chloride), copolymers of acrylamide and dimethyldiallyammonium chloride, and mixtures thereof.

The hair treatment compositions according to the instant disclosure typically include about 0.1 to about 5 wt. % of one or more cationic conditioning polymers, based on the total weight of the hair treatment composition. The hair treatment compositions may include about 0.1 to about 4 wt. %, about 0.1 to about 3 wt. %, about 0.1 to about 2 wt. %, about 0.1 to about 1.5 wt. %, about 0.2 to about 5 wt. %, about 0.2 to about 4 wt. %, about 0.2 to about 3 wt. %, about 0.2 to about 2 wt. %, about 0.2 to about 1.5 wt. % of the one or more cationic polymers, based on the total weight of the hair treatment composition.

### (II)(k) Miscellaneous Ingredients

The conditioning compositions optionally include or exclude (or are essentially free from) one or more miscellaneous ingredients. Miscellaneous ingredients include those that are compatible with the conditioning compositions and do not disrupt or materially affect the basic and novel properties of the compositions. A nonlimiting list of useful miscellaneous ingredients is set forth above, under the heading, “(I)(h) Miscellaneous Ingredients,” which is incorporated herein by reference. In other words, the one or more miscellaneous ingredients s set forth in Section “(1)(h) Miscellaneous Ingredients” are useful in the conditioning compositions. They are not reiterated here for the sake of brevity.

The total amount of the one or more miscellaneous ingredients in the conditioning compositions, if present, will vary. Nonetheless, in various embodiments, the conditioning compositions include about 0.1 to about 15 wt. % of the one or more miscellaneous ingredients, based on the total weight of the compositions. In further embodiments, the conditioning compositions include about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. % of the one or more miscellaneous ingredients, based on the total weight of the conditioning composition.

### Viscosity

Viscosity measurements of the conditioning composition may be carried out using a Brookfied Viscometer DV-II+ Pro (Model D), Spindle B (Heliopath) at 50 RPM, 1 min., at 25° C. The conditioning compositions typically have a viscosity of about 50,000 to about 250,000 cP at 25° C. The viscosity can also be from about 50,000 to about 200,000 cP, about 50,000 to about 150,000 cP, about 75,000 to about 250,000 cP, about 75,000 to about 200,000 cP, about 75,000 to about 150,000 cP, about 100,000 to about 250,000 cP, about 100,000 to about 200,000 cP, or about 100,000 to about 150,000 cp at 25° C.

pH

The pH of the conditioning composition can vary. Nonetheless, in various embodiments, a pH less than 7 (an acidic pH) is desirable. For example, the pH can be from about 3 to about 6.5, about 3 to about 6, about 3 to about 5.5, about 3 to about 5, about 3 to about 4.5, about 3.2 to about 6.5, about 3.2 to about 6, about 3.2 to about 5.5, about 3.2 to about 5, or about 3.2 to about 4.5, preferably about 3 to about 4.5.

### Embodiments of Conditioning Compositions

In various embodiments, the conditioning composition comprises, consists essentially of, or consists of:
(a) about 1 to about 5 wt. %, preferably about 1.5 to about 4, more preferably about 2 to about 4 wt. % of citric acid, a salt thereof, or a mixture thereof;
(b) about 0.5 to about 5 wt. %, preferably about 1 to about 4, more preferably about 1 to about 3 wt. % of a cyclodextrin, derivative thereof, or combination thereof, preferably selected from α-cyclodextrin, β-cyclodextrin, γ-cyclodextrin, methyl derivatives of α-cyclodextrin, β-cyclodextrin, or γ-cyclodextrin, hydroxypropyl derivatives of α-cyclodextrin, β-cyclodextrin, or γ-cyclodextrin, or mixtures thereof, more preferably wherein the cyclodextrin, derivative thereof, or combination thereof comprises or consists of β-cyclodextrin;
   wherein a combined total of (a) and (b) is about 2 to about 12 wt. %, preferably about 2 to about 10 wt. %, more preferably about 3 to about 8 wt. %, and even more preferably about 3 to about 6 wt. %;
   wherein preferably the citric acid, salts thereof, or combination thereof of (a) and the cyclodextrin, derivative thereof, or combination thereof of (b) are individually combined with each other to form a composition in which the cyclodextrin, derivative thereof, or combination thereof of (b) is dissolved in the citric acid, salt thereof, or combination thereof of (a) before subsequently being combined with additional components of the conditioning composition;
(c) about 1 to about 10 wt. %, preferably about 1 to about 8 wt. %, more preferably about 2 to about 6 wt. % of one or more cationic surfactants, preferably one or more mono-alkyl trimonium halide compounds, for example, more mono-alkyl trimonium halide compounds selected from cetrimonium chloride, steartrimonium chloride, behentrimonium chloride, cocotrimonium chloride, cocamidopropyltrimonium chloride. Preferred are cetrimonium chloride, steartrimonium chloride and behentrimonium chloride, even more preferably wherein the one or more cationic surfactants includes behentrimonium chloride, optionally in combination with cetrimonium chloride;
(d) about 0.1 to about 10 wt. %, preferably about 0.5 to about 8, more preferably about 1 to about 5 of one or more nonionic surfactants or emulsifiers, preferably wherein the one or more nonionic surfactants or emulsifiers are selected from alkoxylated fatty alcohols, fatty acid esters of polyoxyethylene glycol, ethoxylated mono or diglycerides, sorbitan esters, ethoxylated sorbitan esters, fatty acid glycol esters, ethylene oxide, alkyl(ether)phosphates, alkylpolyglucosides, or a mixture thereof, more preferably one or more alkyl or polyalkyl ethers of poly(ethylene oxide) containing at least one C8-C30 alkyl radical, with a number of ethylene oxide (EO) units ranging from 2 to 200, in particular, selected from laureth-3, laureth-4, laureth-7, laureth-23, ceteth-5, ceteth-7, ceteth-15, ceteth-23, oleth-5, oleth-7, oleth-10, oleth-12, oleth-20, oleth-50, phytosterol 30 EO, steareth-6, steareth-20, steareth-21, steareth-40, steareth-100, beheneth 100, ceteareth-7, ceteareth-10, ceteareth-15, ceteareth-25, pareth-3, pareth-23, C12-15 pareth-3, C12-13 pareth-4, C12-13 pareth-23, trideceth-3, trideceth-4, trideceth-5, trideceth-6, trideceth-7 and trideceth-10, or mixtures thereof;
(e) about 1 to about 15 wt. %, preferably about 2 to about 12, more preferably about 4 to about 10 wt. % of one or more fatty alcohols, preferably one or more fatty alcohols selected from fatty alcohols have from 12 to 24 carbon atoms, more preferably one or more fatty alcohols selected from one or more solid fatty alcohols, for example, cetyl alcohol, stearyl alcohol, behenyl alcohol and mixtures thereof;
(f) about 2 to about 18 wt. %, preferably about 2 to about 12 wt. %, more preferably about 3 to about 8 wt. % of one or more amino-functionalized silicones, preferably one or more amino-functionalized silicones selected from amodimethicone, bis-hydroxy/methoxy amodimethicone, bis-cetearyl amodimethicone, bis(C13-15 alkoxy) PG amodimethicone, aminopropyl phenyl trimethicone, aminopropyl dimethicone, bis-amino PEG/PPG-41/3 aminoethyl PG-propyl dimethicone, or a mixture thereof, more preferably wherein the one or more amino-functionalized silicones includes amodimethicone or is amodimethicone;
(g) optionally, about 0.1 to about 14 wt. %, preferably about 1 to about 12 wt. %, more preferably about 2 to about 8 wt. % of one or more water-soluble solvents, preferably wherein the one or more water soluble solvents are selected from glycerin, C1-C6 mono-alcohols, polyols (polyhydric alcohols), glycols, or a mixture thereof, more preferably wherein the one or more water soluble solvents are selected from glycerin, glycols (e.g., ethylene glycol, propylene glycol, butylene glycol, pentylene glycol, caprylyl glycol, etc.), or a combination thereof;
(h) about 60 to about 85 wt. %, preferably about 65 to about 80 wt. %, more preferably about 70 to about 80 wt. % of water;
(i) optionally, about 0.1 to about 12 wt. %, preferably about 0.5 to about 10 wt. %, more preferably about 1 to about 6 wt. % of one or more non-silicone-based fatty compounds, preferably one or more non-silicone-based fatty compounds selected from oils, waxes, linear or branched alkanes, fatty esters, esters of fatty acids, esters of fatty alcohols, cetyl esters, triglycerides, or a mixture thereof, more preferably wherein the one or more non-silicone-based fatty compounds is selected from oil, cetyle esters, or a mixture thereof;
(j) optionally, about 0.01 to about 5 wt. %, preferably about 0.1 to about 4 wt. %, more preferably about 0.1 to about 3 wt. % of one or more cationic conditioning polymers, preferably one or more cationic conditioning polymers selected from cationic guar gum derivatives, such as cationic guar or cationic guar derivatives (e.g., guar hydroxypropyltrimonium chloride), cationic proteins or derivative thereof (hydroxypropyltrimonium hydrolyzed wheat protein), polyquaterniums, or a combination thereof;
(k) optionally, about 0.01 to about 10 wt. %, preferably about 0.1 to about 8 wt. %, more preferably about 1 to about 5 wt. % of one or more miscellaneous ingredients, preferably selected from preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, silica, particular materials, etc.), emollients, composition colorants, or a mixture thereof;
   wherein all weight percentages are based on a total weight of the conditioning composition.

The pH of the composition can vary. Nonetheless, in various embodiments, a pH less than 7 (an acidic pH) is desirable. For example, the pH can be from about 3 to about 6.5, about 3 to about 6, about 3 to about 5.5, about 3 to about 5, about 3 to about 4.5, about 3.5 to about 6.5, about 3.5 to about 6, about 3.5 to about 5.5, about 3.5 to about 5, or about 3.5 to about 4.5.

The total combined amount of the citric acid, salts thereof, or combination of (a) and the cyclodextrin or derivatives of (b) will vary. Nonetheless, in various embodiments the total combined amount of the citric acid, salts thereof, or combination of (a) and the cyclodextrin and derivatives of (b) is about 1 to about 12 wt. %, based on a total weight of the hair treatment composition. In further embodiments, the total combined amount of the citric acid, salts thereof, or combination of (a) and the cyclodextrin and derivatives of (b) is about 1 to about 10 wt. %, about 1 to about 8 wt. %, about 1 to about 6 wt. %, about 1 to about 5 wt. %, about 2 to about 12 wt. %, about 2 to about 10 wt. %, about 2 to about 8 wt. %, about 2 to about 6 wt. %, about 2 to about 5 wt. %, about 3 to about 12 wt. %, about 3 to about 10 wt. %, about 3 to about 8 wt. %, about 3 to about 6 wt. %, about 3 to about 5 wt. %, or about 1 wt. %, 2 wt. %, 3 wt. %, 4 wt. %, 5 wt. %, 6 wt. %, 7 wt. %, or 8 wt. %, based on a total weight of the hair treatment composition.

The weight ratio of the citric acid, salts thereof, or combination of (a) to the cyclodextrin or derivatives of (b) will vary. Nonetheless, in various embodiments, the citric acid, salts thereof, or combination of (a) and the cyclodextrin or derivatives of (b) are in a weight ratio of about of about 8:1 to about 1:2((a):(b)). In further embodiments, the citric acid, salts thereof, or combination of (a) and the cyclodextrin or derivatives of (b) are in a weight ratio of 6:1 to about 1:2, about 5:2 to about 1:2, about 4:1 to about 1:2, about 3:1 to about 1:2, about 2:1 to about 1:2, about 8:1 to about 1:1, about 6:1 to about 1:1, about 5:1 to about 1:1, about 4:1 to about 1:1, about 3:1 to about 1:1, about 2:1 to about 1:1, about 1.2:1, about 1.3:1, about 1.4:1, about 1.5:1, about 1.6:1, or about 1.8:1 ((a):(b)).

The mole ratio of the citric acid, salts thereof, or combination of (a) to the cyclodextrin or derivatives of (b) will vary. Nonetheless, in various embodiments, the citric acid, salts thereof, or combination of (a) and the cyclodextrin or derivatives of (b) are in a mole ratio of about 20:1 to about 3:1. In further embodiments, the citric acid, salts thereof, or combination of (a) and the cyclodextrin or derivatives of (b) are in a mole ratio of about 18:1 to about 3:1, about 15:1 to about 3:1, about 20:1 to about 5:1, about 18:1 to about 5:1, about 15:1 to about 5:1, about 20:1 to about 8:1, about 18:1 to about 8:1, about 15:1 to about 8:1, about 20:1 to about 10:1, about 18:1 to about 10:1, about 15:1 to about 10:1, about 14:1, about 13:1, about 12:1, or about 11:1.

Application and Processing with Conditioning Composition

The conditioning composition can be applied to wet, damp, or dry hair. Nonetheless, the conditioning composition is typically applied to damp hair, for example, damp hair that has been rinsed shortly before treatment with the conditioning composition. For example, the hair has typically been rinsed after treatment with the alkaline composition. Typically, the conditioning composition is applied to the hair 1 hour after completion of treatment with the alkaline composition. Nonetheless, often the conditioning composition is applied more quickly after treating the hair with the alkaline composition. For example, the conditioning composition may be applied to the hair within 30 minutes, within 15 minutes, within 10 minutes, or within 5 minutes after completion of treatment with the alkaline composition.

The conditioning composition is distributed throughout the hair and allowed to remain on the hair (process) for a period of time. Typically, the conditioning composition is allowed to remain on the hair for a period of time sufficient to condition the hair, i.e., impart a conditioning effect to the hair. For example, the conditioning composition may be allowed to remain on the hair for about 1 minutes to about 60 minutes, about 1 minute to about 45 minutes, about 1 minute to about 30 minutes, about 1 minute to about 20 minutes, about 1 minute to about 15 minutes about 2 minutes to about 60 minutes, about 2 minute to about 45 minutes, about 2 minute to about 30 minutes, about 2 minute to about 20 minutes, about 2 to about 15 minutes, about 3 minutes to about 60 minutes, about 3 minute to about 45 minutes, about 3 minute to about 30 minutes, about 3 minute to about 20 minutes, about 3 minutes to about 15 minutes, about 5 minutes to about 60 minutes, about 5 minute to about 45 minutes, about 5 minute to about 30 minutes, about 5 minute to about 20 minutes, or about 5 minutes to about 15 minutes.

After remaining on the hair for the period of time, the conditioning composition is typically rinsed from the hair with water. Next, the hair is treated with the leave-on composition, described in more detail below. Typically, the hair is treated with the leave-on compositions shortly after rinsing the conditioning composition from the hair, for example, within 1 hour. In various embodiments, the hair is treated with the leave-on composition within about 45 minutes, about 30 minutes, about 15 minutes, about 10 minutes, or about 5 minutes of rinsing the conditioning composition from the hair.

### Leave On Composition

### (III)(a) Silicones

Silicones can be referred to as silicone oils. Nonlimiting examples of silicones include dimethicone, dimethiconol, dimethiconol, cyclomethicone, polysilicone-11, phenyl trimethicone, trimethylsilylamodimethicone, and stearoxytrimethylsilane. In a preferred embodiment, the one or more silicones are chosen from non-volatile silicon oils. Useful silicone oils include polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates. Other examples of silicone oils that may be mentioned include volatile linear or cyclic silicones, such as those with a viscosity 8 centistokes (8×106 m2/s) and/or containing from 2 to 7 silicon atoms. These silicones optionally comprise alkyl or alkoxy groups containing from 1 to 10 carbon atoms. Non-limiting examples of volatile silicone oils include octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, or mixtures thereof.

Nonlimiting examples of quaternized or quaternizable polysiloxanes include silicone quaternium 22 (e.g., ABIL T QUAT 60), silicone quaternium 12 (PECOSIL CA-1240), amodimethicone (e.g., DOW CORNING 2-8566 AMINO FLUID), bis-cetearyl amodimethicone (e.g., SILSOFT AX), bis-amino PEG/PPG-41/3 aminoethyl PG-propyl dimethicone (e.g., SILSOFT A-843), PEG-40/PPG-8 methylaminopropyl hydroxypropyl dimethicone copolymer (e.g., SILSOFT A+), silicone quaterium 16 (and) undeceth-11 (and) butyloctanol (and) undeceth-5 (e.g., DOW CORNING 5-7113 SILICONE QUAT MICROEMULSION), and bis-isobutyl/PEG/PPG-20/35/amodimethicone copolymer (e.g., DOW CORNING CE 8401 EMULSION). A preferred quaternized or quaternizable polysiloxane is bis-cetearyl amodimethicone.

In certain embodiments, at least one of the one or more silicones is an amino functionalized silicone. The term “amino-functionalized silicone” is intended to indicate any silicone comprising at least one primary, secondary or tertiary amine or a quaternary ammonium group without piperidinyl groups. The term “amino-functionalized silicone” is interchangeable with the term “amino silicone.” In some instances, the amino-functionalized silicones are alkoxylated and/or hydroxylated amino silicones. Nonlimiting examples include amodimethicone, bis-hydroxy/methoxy amodimethicones, bis-cetearyl amodimethicone, amodimethicone, bis(C13-15 alkoxy) PG amodimethicones, aminopropyl phenyl trimethicones, aminopropyl dimethicones, bis-amino PEG/PPG-41/3 aminoethyl PG-propyl dimethicones, caprylyl methicones, and a mixture thereof. Aminopropyl dimethicone and amodimethicone are particularly preferred amino functionalized silicones.

A more exhaustive but non-limiting description of amino-functionalized silicones is set forth above, under the heading “(i)(g) Amino-Functionalized Silicone,” and is incorporated herein by reference in its entirety.

Notwithstanding the above, nonlimiting examples of amino-functionalized silicones useful in the leave-on compositions include aminopropyl dimethicone, amodimethicone, quaternium 80, silicone quaternium-1, silicone quaternium-2, silicone quaternium-2 panthenol succinate, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, silicone quaternium-16, silicone quaternium-16/Glycidoxy Dimethicone Crosspolymer, silicone quaternium-17, silicone quaternium-18, silicone quaternium-20 and silicone quaternium-21. Preferred are quaternium 80, silicone quaternium-16, silicone quaternium-18, silicone quaternium-1, silicone quaternium-2, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, silicone quaternium-17, silicone quaternium-20 and silicone quaternium-21. More preferred are quaternium 80, silicone quaternium-16, silicone quaternium-18, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, and silicone quaternium-17. Preferred are quaternium 80, silicone quaternium-16, silicone quaternium-18, silicone quaternium-15, bis-hydroxy/methoxy amodimethicones, bis-cetearyl amodimethicone, bis(C13-15 alkoxy) PG amodimethicones, aminopropyl phenyl trimethicones, bis-amino PEG/PPG-41/3 aminoethyl PG-propyl dimethicones, caprylyl methicones, and a mixture thereof. A particularly preferred amino-functionalized silicone is aminopropyl dimethicone.

The total amount of the one or more silicones, preferably one or more amino-functionalized silicones, in the leave-on compositions will vary. Nonetheless, in various embodiments, the total amount of the one or more silicones, preferably one or more amino-functionalized silicones in the leave-on compositions, is from about 0.1 to about 15 wt. %, based on the total weight of the composition. In further embodiments, the total amount of the one or more silicones, preferably amino-functionalized silicones, in the leave-on composition is from about 0.1 to about 12 wt. %, about 0.1 to about 10 wt. %, about 0.1 to about 8 wt. %, about 1 to about 15 wt. %, about 1 to about 12 wt. %, about 1 to about 10 wt. %, about 1 to about 8 wt. %, about 2 to about 15 wt. %, about 2 to about 12 wt. %, about 2 to about 10 wt. %, about 2 to about 8 wt. %, about 3 to about 15 wt. %, about 3 to about 12 wt. %, about 3 to about 10 wt. %, about 3 to about 8 wt. %, about 1 wt. %, about 2 wt. %, about 3 wt. %, about 4 wt. %, about 5 wt. %, about 6 wt. %, about 7 wt. %, about 8 wt. %, or about 10 wt. %, based on a total weight of the leave-on composition.

### (III)(b) Film-Forming Polymers

The one or more film-forming polymer may include one or more nonionic film forming polymers, one or more amphoteric hair film forming polymers, or a combination thereof.

Non-limiting examples of nonionic film-forming polymers include vinylpyrrolidone homopolymers; copolymers of vinylpyrrolidone and of vinyl acetate; polyalkyloxazolines; vinyl acetate homopolymers; copolymers of vinyl acetate and of acrylic ester; copolymers of vinyl acetate and of ethylene; copolymers of vinyl acetate and of maleic ester; copolymers of polyethylene and of maleic anhydride; alkyl acrylate homopolymers and alkyl methacrylate homopolymers; acrylic ester copolymers; copolymers of acrylonitrile and of a non-ionic monomer; and a mixture thereof. In some cases, particularly useful nonionic film-forming polymers include vinylpyrrolidone homopolymers and copolymers of vinylpyrrolidone and of vinyl acetate, for example, polyvinylpyrrolidone/vinyl acetate (VP/VA) copolymer. In some instances, nonionic film forming polymers of the present disclosure are selected from the group consisting of vinylpyrrolidone homopolymers, copolymers of vinylpyrrolidone and of vinyl acetate, and a mixture thereof. Vinylpyrrolidone homopolymers (INCI name: polyvinylpyrrolidone) are commercially available from Ashland Specialty Ingredients under the tradename PVP K. Copolymers of vinylpyrrolidone and of vinyl acetate (INCI name: VP/VA copolymer) are commercially available from BASF under the tradename Luviskol VA.

Nonlimiting examples of amphoteric film-forming polymers include:
(1) polymers resulting from the copolymerization of a monomer derived from a vinyl compound bearing a carboxylic group such as, more particularly, acrylic acid, methacrylic acid, maleic acid, α-chloroacrylic acid, and a basic monomer derived from a substituted vinyl compound containing at least one basic atom, such as, more particularly, dialkylaminoalkyl methacrylates and acrylates, dialkylaminoalkylmethacrylamides and -acrylamides;
(2) polymers containing units derived from:
   a) at least one monomer chosen from acrylamides and methacrylamides substituted on the nitrogen with an alkyl radical,
   b) at least one acidic comonomer containing one or more reactive carboxylic groups, and
   c) at least one basic comonomer such as esters containing primary, secondary, tertiary and quaternary amine substituents of acrylic and methacrylic acids and the product of quaternization of dimethylaminoethyl methacrylate with dimethyl or diethyl sulphate.

The N-substituted acrylamides or methacrylamides which may be useful are groups in which the alkyl radicals contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers include acrylic acid, methacrylic acid, crotonic acid, itaconic acid, maleic acid and fumaric acid and alkyl monoesters, having 1 to 4 carbon atoms, of maleic or fumaric acids or anhydrides.

In some cases, preferred basic comonomers are aminoethyl, butylaminoethyl, N,N′-dimethylaminoethyl and N-tert-butylaminoethyl methacrylates. Octylacrylamide/acrylates/butylaminoethyl methacrylate copolymer such as the products sold under the name Amphomer or Balance 47 (formerly Lovocryl 47) by the company Akzo Nobel can be used.
(3) crosslinked and alkylated polyamino amides partially or totally derived polyamino amides.
(4) polymers containing zwitterionic units of formula:

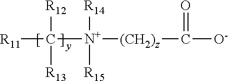

in which R₁₁ denotes a polymerizable unsaturated group, such as an acrylate, methacrylate, acrylamide or methacrylamide group, y and z represent an integer from 1 to 3, R₁₂ and R₁₃ represent a hydrogen atom or methyl, ethyl or propyl, and R₁₄ and R₁₅ represent a hydrogen atom or an alkyl radical such that the sum of the carbon atoms in R₁₄ and R₁₅ does not exceed 10.

The polymers comprising such units can also contain units derived from non-zwitterionic monomers such as dimethyl or diethylaminoethyl acrylate or methacrylate or alkyl acrylates or methacrylates, acrylamides or methacrylamides or vinyl acetate:

By way of example, mention may be made of the copolymer of methyl methacrylate/dimethyl carboxymethylammonio methyl ethylmethacrylate.
(5) Polymers derived from chitosan.
(6) Polymers derived from the N-carboxyalkylation of chitosan, such as N-carboxymethylchitosan or N-carboxybutylchitosan sold under the name “Evalsan” by the company Jan Dekker.
(7) Polymers corresponding to the general formula:

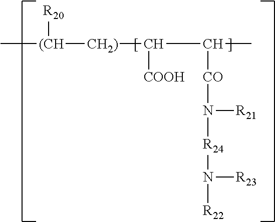

in which R₂₀ represents a hydrogen atom, a CH₃O, CH₃CH₂O or phenyl radical, R₂₁ denotes hydrogen or a lower alkyl radical such as methyl or ethyl, R₂₂ denotes hydrogen or a lower alkyl radical such as methyl or ethyl, R₂₃ denotes a lower alkyl radical such as methyl or ethyl or a radical corresponding to the formula: —R.sub.24-N(R₂₂).sub.2, R₂₄ representing a —CH₂—CH₂, —CH₂—CH₂—CH₂— or —CH₂—CH(CH₃)-group, R₂₂ having the meanings mentioned above, as well as the higher homologues of these radicals and containing up to 6 carbon atoms.
(8) Amphoteric polymers of the type -D-X-D-X chosen from:
   a) polymers obtained by the action of chloroacetic acid or sodium chloroacetate on compounds containing at least one unit of formula:

-D-X-D-X-D- (I)
where D denotes a radical

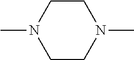

and X denotes the symbol E or E′, E or E′, which may be identical or different, denotes a divalent radical which is an alkylene radical containing a straight or branched chain containing up to 7 carbon atoms in the main chain, which is unsubstituted or substituted with hydroxyl groups and which can contain, in addition to the oxygen, nitrogen and sulphur atoms, 1 to 3 aromatic and/or heterocyclic rings; the oxygen, nitrogen and sulphur atoms being present in the form of ether, thioether, sulphoxide, sulphone, sulphonium, alkylamine or alkenylamine groups, hydroxyl, benzylamine, amine oxide, quaternary ammonium, amide, imide, alcohol, ester and/or urethane groups.
   b) Polymers of formula:

-D-X-D-X— (I′)
in which D denotes a radical

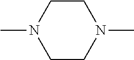

and X denotes the symbol E or E′ and at least once E′; E having the meaning given above and E′ is a divalent radical which is an alkylene radical with a straight or branched chain having up to 7 carbon atoms in the main chain, which is unsubstituted or substituted with one or more hydroxyl radicals and containing one or more nitrogen atoms, the nitrogen atom being substituted with an alkyl chain which is optionally interrupted by an oxygen atom and necessarily containing one or more carboxyl functions or one or more hydroxyl functions and betainized by reaction with chloroacetic acid or sodium chloroacetate.
(9) (C1-C5)alkyl vinyl ether/maleic anhydride copolymers, the maleic anhydride being partially modified by semiamidation with an N,N-dialkylaminoalkylamine such as N,N-dimethyl-aminopropylamine or by semiesterification with an N,N-dialkanolamine. These copolymers can also contain other vinyl comonomers such as vinylcaprolactam.

The amphoteric film-forming polymers which that may be particularly useful are those of family (3), such as the copolymers whose CTFA name is octylacrylamide/acrylates/butylaminoethyl methacrylate copolymer, such as the products sold under the names Amphomer LV 71 by the company Akzo Nobel.

In some instances, the one or more amphoteric film-forming polymers are selected from the group consisting of:
(i) polymers resulting from the copolymerization of a monomer derived from a vinyl compound bearing a carboxylic group selected from the group consisting of acrylic acid, methacrylic acid, maleic acid, and α-chloroacrylic acid, and a basic monomer derived from a substituted vinyl compound containing at least one basic atom;
(ii) polymers containing units derived from:
   a) at least one monomer selected from the group consisting of acrylamides and methacrylamides substituted on the nitrogen with an alkyl radical,
   b) at least one acidic comonomer containing one or more reactive carboxylic groups, and
   c) at least one basic comonomer ester containing primary, secondary, tertiary or quaternary amine substituents of acrylic and methacrylic acids or the product of quaternization of dimethylaminoethyl methacrylate with dimethyl or diethyl sulphate;
(iii) crosslinked and alkylated polyamino amides partially or totally derived polyamino amides; and
(vi) copolymers of methyl methacrylate/dimethyl carboxymethyl-ammoniomethylethylmethacrylate.

In a preferred embodiment, the leave-on compositions include one or more the nonionic associative polyurethane thickeners as the one or more film-forming polymers. The nonionic polyurethane/polyethers may have both at least one hydrophilic moiety and at least one hydrophobic moiety. More particularly, said polymers may contain in their chain both hydrophilic sequences most often of a polyoxyethylenated nature and hydrophobic sequences which may be aliphatic linkages alone and/or cycloaliphatic and/or aromatic linkages. In various embodiments, these polyether-polyurethanes comprise at least two lipophilic hydrocarbon chains, having from 6 to 30 carbon atoms, preferably from 6 to 20, separated by a hydrophilic sequence, it being possible for the hydrocarbon chains to be pendent chains or chains at the end of a hydrophilic sequence. In particular, it is possible for one or more pendent chains to be envisaged. In addition, the polymer may comprise a hydrocarbon chain at one end or at both ends of a hydrophilic sequence.

The polyether-polyurethanes may be polyblocks, in particular in triblock form. The hydrophobic sequences may be at each end of the chain (for example: triblock copolymer with hydrophilic central sequence) or distributed both at the ends and in the chain (polyblock copolymers for example). These same polymers may also be in the form of graft units or may be star shaped.

The nonionic polyether/polyurethanes containing a fatty chain may be triblock copolymers whose hydrophilic sequence is a polyoxyethylenated chain comprising from 50 to 1000 oxyethylenated groups. The nonionic polyether-polyurethanes comprise a urethane bond between the hydrophilic sequences, hence the origin of the name. By extension, those whose hydrophilic sequences are linked by other chemical bonds to the hydrophobic sequences are also included among the nonionic polyether-polyurethanes containing a hydrophobic chain.

Nonlimiting examples of nonionic polyether/polyurethanes containing a hydrophobic chain include Rheolate® 205 containing a urea functional group sold by the company RHEOX or else the Rheolates® 208, 204 or 212, as well as Acrysol RM 184®. Additional products include ELFACOS T210® containing a C12-C14 alkyl chain and the product ELFACOS T212® containing a C18 alkyl chain from AKZO. The product DW 1206B® from ROHM & HAAS containing a C20 alkyl chain and with a urethane bond, sold at 20% dry matter content in water, may also be used.

It is also possible to use solutions or dispersions of these polymers in particular in water or in an aqueous-alcoholic medium. By way of examples of such polymers, there may be mentioned Rheolate® 255, Rheolate® 278 and Rheolate® 244 sold by the company RHEOX. It is also possible to use the product DW 1206F and DW 1206J provided by the company ROHM & HAAS.

As the above-described polyether/polyurethanes, mention may be made of polyurethane/polyethers comprising in their chain at least one polyoxyethylenated hydrophilic block and at least one of hydrophobic blocks containing at least one sequence chosen from aliphatic sequences, cycloaliphatic sequences, and aromatic sequences. In various embodiments, it is preferable that the polyurethane/polyethers comprise at least two hydrocarbon-based lipophilic chains having from 8 to 30 carbon atoms, separated by a hydrophilic block, and wherein the hydrocarbon-based chains are chosen from pendent chains and chains at the end of the hydrophilic block.

In a preferred embodiment, use is made a polyurethane/polyether that may be obtained by polycondensation of at least three compounds comprising (i) at least one polyethylene glycol comprising from 150 to 180 mol of ethylene oxide, (ii) a polyoxyethylenated stearyl alcohol comprising 100 mol of ethylene oxide, and (iii) a diisocyanate. Such polyurethane/polyethers are sold especially by the company Element is under the name Rheolate FX 1100@ and Rheoluxe 811®, which is a polycondensate of polyethylene glycol containing 136 mol of ethylene oxide, of stearyl alcohol polyoxyethylenated with 100 mol of ethylene oxide and of hexamethylene diisocyanate (HDI) with a weight-average molecular weight of 40000 (INCI name: PEG-136/Steareth-100/HDI Copolymer).

According to another embodiment, use will be made of a polyurethane/polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate. Such polyurethane/polyethers are sold in particular by the company Rohm & Haas under the names Aculyn 46® and Aculyn 44®.

Aculyn 46® having the INCI name: PEG-150/Stearyl Alcohol/SMDI Copolymer, is a polycondensate of polyethylene glycol comprising 150 or 180 mol of ethylene oxide, of stearyl alcohol and of methylenebis(4-cyclohexyl isocyanate) (SMDI) at 15% by weight in a matrix of maltodextrin (4%) and water (81%) (INCI name: PEG-150/Stearyl Alcohol/SMDI Copolymer). Aculyn 44® (PEG-150/Decyl Alcohol/SMDI Copolymer) is a polycondensate of polyethylene glycol comprising 150 or 180 mol of ethylene oxide, of decyl alcohol and of methylenebis(4-cyclohexyl isocyanate) (SMDI) at 35% by weight in a mixture of propylene glycol (39%) and water (26%) (INCI name: PEG-150/Decyl Alcohol/SMDI Copolymer).

As the associative polyurethanes, it may be preferable to use a compound represented by the following formula (1):

R¹—{(O—R²)ₖ—OCONH—R³[—NHCOO—(R⁴—O)ₙ—R⁵]ₕ}ₘ (1)
wherein R¹ represents a hydrocarbon group, R² and R⁴ independently represent alkylene groups having 2 to 4 carbon atoms, which alkylene groups may be identical or different from each other, or a phenylethylene group, R³ represents a hydrocarbon group, which may optionally have a urethane bond, R₅ represents a branched chain or secondary hydrocarbon group, “m” represents a number of at least 2, “h” represents a number of at least 1, “k” represents a number within the range of 1 to 500, and “n” represents a number within the range of 1 to 200.

The hydrophobically modified polyurethane represented by the general formula (1) shown above is obtained by, for example, reacting at least one polyether polyol that is represented by the formula R¹—[(O—R²)ₖ—OH]ₘ, at least one polyisocyanate that is represented by the formula R³—(NCO)ₕ₊₁, and at least one polymonoalcohol that is represented by the formula HO—(R⁴—O)ₙ—R⁵. In such cases, R¹ to R⁵ in the general formula (1) are determined by the compounds R¹—[(O—R²)ₖ—OH]ₘ, R³—(NCO)ₕ₊₁ and HO—(R⁴—O)ₙ—R⁵. The loading ratios among the three compounds are not limited particularly and should preferably be such that the ratio of the isocyanate group derived from the polyisocyanate to the hydroxyl group derived from the polyether polyol and the polyether monoalcohol is selected within the range of NCO/OH of between 0.8:1 and 1.4:1.

The polyether polyol compound that is represented by the formula R¹—[(O—R²)ₖ—OH]ₘ and that may be used preferably for obtaining the associative thickener represented by the general formula (1) may be obtained from addition polymerization of an m-hydric polyol with an alkylene oxide, such as ethylene oxide, propylene oxide, butylene oxide, or epichlorohydrin, or with styrene oxide, and the like.

The polyols should preferably be di- to octa-hydric polyols. Examples of the di- to octa-hydric polyols include dihydric alcohols, such as ethylene glycol, propylene glycol, butylene glycol, hexamethylene glycol, and neopenthyl glycol; trihydric alcohols, such as glycerol, trioxy isobutane, 1,2,3-butanetriol, 1,2,3-pentanetriol, 2-methyl-1,2,3-propanetriol, 2-methyl-2,3,4-butanetriol, 2-ethyl-1,2,3-butanetriol, 2,3,4-pentanetriol, 2,3,4-hexanetriol, 4-propyl-3,4,5-heptanetriol, 2,4-dimethyl-2,3,4-pentanetriol, pentamethylglycerol, pentaglycerol, 1,2,4-butanetriol, 1,2,4-pentanetriol, trimethylolethane, and trimethylolpropane; tetrahydric alcohols, such as pentaerythritol, 1,2,3,4-pentanetetrol, 2,3,4,5-hexanetetrol, 1,2,4,5-pentanetetrol, and 1,3,4,5-hexanetetrol; pentahydric alcohols, such as adonitol, arabitol, and xylitol; hexahydric alcohols, such as dipentaerythritol, sorbitol, mannitol, and iditol; and octahydric alcohols, such as sucrose.

Also, R² is determined by the alkylene oxide, styrene oxide, or the like, which is subjected to the addition. Particularly, for availability and excellent effects, an alkylene oxide having 2 to 4 carbon atoms, or styrene oxide is preferable.

The alkylene oxide, styrene oxide, or the like, to be subjected to the addition may be subjected to single polymerization, or random polymerization or block polymerization of at least two members. The procedure for the addition may be a conventional procedure. Also, the polymerization degree k may be selected within the range of 0 to 1,000, preferably within the range of 1 to 500, and more preferably within the range of 10 to 200. Further, the ratio of the ethylene group occupying R2 should preferably be within the range of 50 to 100 mass % with respect to the total quantity of R2. In such cases, the associative thickener appropriate for the purposes of the present invention is obtained.

Furthermore, the molecular weight of the polyether polyol compound that is represented by the formula R¹—[(O—R²)ₖ—OH]ₘ, should preferably be selected within the range of 500 to 100,000, and should more preferably be selected within the range of 1,000 to 50,000.

The polyisocyanate that is represented by the formula R³—(NCO)ₕ₊₁ and that may be used preferably for obtaining the hydrophobically modified polyether urethane represented by the general formula (1) employed in accordance with the present invention is not limited particularly in so far as the polyisocyanate has at least two isocyanate groups in the molecule. Examples of the polyisocyanates include aliphatic diisocyanates, aromatic diisocyanates, alicyclic diisocyanates, biphenyl diisocyanate, phenylmethane diisocyanate, phenylmethane triisocyanate, and phenylmethane tetraisocyanate.

Also, it is possible to employ dimers and trimers (isocyanurate bonds) of the above-enumerated polyisocyanates. Further, it is possible to employ biuret obtained by a reaction with an amine.

Furthermore, it is possible to employ a polyisocyanate having a urethane bond obtained by a reaction of the aforesaid polyisocyanate compound and a polyol. As the polyol, di- to octa-hydric polyols are preferable, and the above-enumerated polyols are preferable. In cases where a tri- or higher-hydric polyisocyanate is used as the polyisocyanate that is represented by the formula R3-(NCO)ₙ+1, it is preferable to employ the aforesaid polyisocyanate having the urethane bond.

The polyether monoalcohol that is represented by the formula HO—(R⁴—O)ₙ—R⁵ and that may be used preferably for obtaining the hydrophobically modified polyether urethane represented by the general formula (1) employed in accordance with the present invention is not limited particularly in so far as the polyether monoalcohol is a polyether of a straight chain, branched chain, or secondary monohydric alcohol. The polyether monoalcohol may be obtained by addition polymerization of the straight chain, branched chain, or secondary monohydric alcohol with an alkylene oxide, such as ethylene oxide, propylene oxide, butylene oxide, or epichlorohydrin, or with styrene oxide, and the like.

The compound represented by the general formula (1) may be produced by, for example, heating at a temperature of 80 to 90° C. for 1 to 3 hours and thereby causing a reaction to occur in the same manner as that in the ordinary reaction of a polyether and an isocyanate.

As the compound represented by the general formula (1), polyethyleneglycol-240/decyltetradeceth-20/hexamethylene diisocyanate copolymer is preferable. The polyethyleneglycol-240/decyltetradeceth-20/hexamethylene diisocyanate copolymer is referred to also as PEG-240/HDI copolymer bis-decyltetradeceth-20 ether.

In various embodiments, it is preferable that the nonionic associative polyurethane thickener be selected from steareth-100/PEG-136/HDI copolymer sold by the company Rheox under the name of Rheolate FX 1100, PEG-240/HDI copolymer bis-decyltetradeceth-20 ether sold by the company Asahi Denka under the name of Adekanol GT-700, and mixtures thereof.

The amount of the one or more film-forming polymers, if present, will vary. Nonetheless, in various embodiments, the hair styling composition includes about 0.01 to about 10 wt. % of the one or more film-forming polymers, preferably one or more nonionic film-forming polymers, based on a total weight of the hair styling composition. In further embodiments, the hair styling composition includes about 0.01 to about 8 wt. %, about 0.01 to about 6 wt. %, about 0.01 to about 5 wt. %, about 0.01 to about 3 wt. %, about 0.05 to about 10 wt. %, about 0.05 to about 8 wt. %, about 0.05 to about 6 wt. %, about 0.05 to about 5 wt. %, about 0.05 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %. about 0.5 wt. %, about 0.6 wt. %, about 0.8 wt. %, about 0.9 wt. %, about 1 wt. %, or about 1.5 wt. % of the one or more film-forming polymers, preferably nonionic film forming polymers such as nonionic associative polyurethane thickener, based on a total weight of the hair styling composition.

### (III)(c) Water

The leave-on composition is an aqueous composition, typically containing a substantial amount of water. For example, in various embodiments, the leave-on composition includes about 50 to about 90 wt. % of water, based on a total weight of the composition. In further embodiments, the leave-on compositions include about 60 to about 90 wt. %, about 70 to about 90 wt. %, about 75 to about 90 wt. %, about 50 to about 85 wt. %, about 60 to about 85 wt. %, about 70 to about 85 wt. %, about 75 to about 85 wt. %, about 50 to about 80 wt. %, about 60 to about 80 wt. %, about 70 to about 80 wt. %, about 75 to about 80 wt. %, about 65 to about 85 wt. %, about 70 wt. %, about 72 wt. %, about 75 wt. %, about 76 wt. %, about 78 wt. %, about 80 wt. %, or about 82 wt. %, based on the total weight of the compositions.

### (III)(d) Non-Silicone Fatty Compound

A nonlimiting list of useful non-silicone fatty compound ds is set forth above, under the heading “(I)(c) Non-Silicone Fatty Compounds” for the alkaline compositions, which is incorporated herein by reference. In other words, the Non-Silicone Fatty Compounds set forth in Section “(I)(c) Non-Silicone Fatty Compounds” are useful in the leave-on composition. They are not exhaustively reiterated here for the sake of brevity.

In a preferred embodiment, the leave-on compositions include one or more non-silicone-based fatty compounds selected from oils, waxes, linear or branched alkanes, fatty esters, esters of fatty acids, esters of fatty alcohols, cetyl esters, triglycerides, or a mixture thereof.

The total amount of the one or more non-silicone-based fatty compounds in the leave-on compositions, if present, will vary. Nonetheless, in various embodiments, the leave-on compositions include about 0.1 to about 20 wt. % of the one or more non-silicone-based fatty compounds, based on the total weight of the compositions. In further embodiments, the compositions include about 0.1 to about 15 wt. %, about 0.1 to about 12 wt. %, about 0.1 to about 10 wt. %, about 0.1 to about 8 wt. %, about 0.5 to about 20 wt. %, about 0.5 to about 15 wt. %, about 0.5 to about 12 wt. %, about 0.5 to about 10 wt. %, about 0.5 to about 8 wt. %, about 1 to about 20 wt. %, about 1 to about 15 wt. %, about 1 to about 12 wt. %, about 1 to about 10 wt. %, about 1 to about 8 wt. %, about 2 to about 20 wt. %, about 2 to about 15 wt. %, about 2 to about 12 wt. %, about 2 to about 10 wt. %, about 2 to about 8 wt. %, about 3 to about 20 wt. %, about 3 to about 15 wt. %, about 3 to about 12 wt. %, about 3 to about 10 wt. %, about 3 to about 8 wt. %, about 5 to about 20 wt. %, about 5 to about 15 wt. %, about 5 to about 12 wt. %, about 5 to about 10 wt. %, about 5 to about 8 wt. %, about 5 wt. %, about 6 wt. %, about 7 wt. %, about 8 wt. %, about 9 wt. %, or about 10 wt. %, based on the total weight of the compositions.

### (III)(e) Polysaccharide Thickening Agents

A nonlimiting list of polysaccharide thickening agents useful in the leave-on compositions is set forth above, under the heading “(I)(f) Polysaccharide Thickening Agents” for the alkaline compositions, which is incorporated herein by reference. In other words, the polysaccharide thickening agents set forth in Section “(I)(f) Polysaccharide Thickening Agents” are useful in leave-on compositions. They are not exhaustively reiterated here for the sake of brevity. Nonetheless, in a preferred embodiments, the one or more polysaccharide thickening agents included in the leave-on composition comprises or are selected from cellulose-based polymers such as cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate; alkyl-substituted celluloses, such as, cellulose ethers; scleroglucans; and gums such as acacia, agar, algin, alginic acid, ammonium alginate, amylopectin, calcium alginate, calcium carrageenan, carnitine, carrageenan, dextrin, gelatin, gellan gum, guar gum, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, biosacharide gum, and mixtures thereof. In a preferred embodiment, the one or more polysaccharide thickening agent comprises or consists of sclerotium gum, guar gum, hydroxypropyl guar, or a combination thereof.

The total amount of the one or more polysaccharide thickening agents in the leave-on compositions will vary. Nonetheless, in various embodiments, the compositions include about 0.1 to about 8 wt. % of the one or more polysaccharide thickening agents, based on the total weight of the composition. In further embodiments, the leave-on compositions include about 0.1 to about 6 wt. %, about 0.1 to about 5 wt. %, about 0.1 to about 4 wt. %, about 0.1 to about 3 wt. %, about 0.1 to about 2 wt. %, about 0.2 to about 8 wt. %, about 0.2 to about 6 wt. %, about 0.2 to about 5 wt. %, about 0.2 to about 4 wt. %, about 0.2 to about 3 wt. %, about 0.2 to about 2 wt. %, about 0.3 to about 8 wt. %, about 0.3 to about 6 wt. %, about 0.3 to about 5 wt. %, about 0.3 to about 5 wt. %, about 0.3 to about 4 wt. %, about 0.3 to about 3 wt. %, about 0.3 to about 2 wt. %, about 0.2 wt. %, about 0.3 wt. %, about 0.4 wt. %, about 0.5 wt. %, about 0.6 wt. %, about 0.7 wt. %, or about 0.8 wt. % of the one or more polysaccharide thickening agents, based on the total weight of the composition.

### (III)(f) Water-Soluble Organic Solvent

The term “water soluble organic solvent” is interchangeable with the terms “water soluble solvent” and “water-miscible solvent” and means a compound that is liquid at 25° C. and at atmospheric pressure (760 mmHg), and it has a solubility of at least 50% in water under these conditions. In some cases, the water-soluble solvents has a solubility of at least 60%, 70%, 80%, or 90%. Non-limiting examples of water-soluble solvents include, for example, organic solvents selected from glycerin, alcohols (for example C₁₋₈, or C₁₋₄ alcohols), polyols (polyhydric alcohols), glycols, and a mixture thereof.

Nonlimiting examples of water-soluble organic solvents. Non-limiting examples of water-soluble organic solvents include, for example, organic solvents selected from glycerin, alcohols (for example, C₁₋₁₀, C₁₋₈, or C₁₋₄ alcohols), polyols (polyhydric alcohols), glycols, and a mixture thereof. Nonlimiting examples of monoalcohols and polyols include ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin.

Further non-limiting examples of water soluble organic solvents include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

In a preferred embodiment, the composition include one or more glycols selected from glycerin, propylene glycol, butylene glycol, pentylene glycol, hexylene glycol, caprylyl glycol, dipropylene glycol, and mixtures thereof.

The total amount of the one or more water soluble solvents in the compositions, if present, will vary. Nonetheless, in various embodiments, the compositions include about 0.1 to about 20 wt. % of the one or more water soluble solvents, based on the total weight of the compositions. In further embodiments, the compositions include about 0.1 to about 15 wt. %, about 0.1 to about 10 wt. %, about 0.5 to about 20 wt. %, about 0.5 to about 15 wt. %, about 0.5 to about 10 wt. %, about 1 to about 20 wt. %, about 1 to about 15 wt. %, about 1 to about 10 wt. %, about 2 to about 20 wt. %, about 2 to about 15 wt. %, about 2 to about 10 wt. %, about 5 to about 20 wt. %, about 5 to about 15 wt. %, or about 5 to about 10 wt. %, based on the total weight of the compositions.

### (III)(g) Miscellaneous Ingredient

The leave-on compositions optionally include or exclude (or are essentially free from) one or more miscellaneous ingredients. Miscellaneous ingredients are ingredients that are compatible with the leave-on treatment compositions and do not disrupt or materially affect the basic and novel properties of the compositions. A nonlimiting list of useful miscellaneous ingredients is set forth above, under the heading, “(I)(h) Miscellaneous Ingredients,” which is incorporated herein by reference. In other words, the one or more miscellaneous ingredients s set forth in Section “(1)(h) Miscellaneous Ingredients” are useful in the conditioning compositions. They are not reiterated here for the sake of brevity.

The total amount of the one or more miscellaneous ingredients in the leave-on compositions, if present, will vary. Nonetheless, in various embodiments, the leave-on compositions include about 0.1 to about 15 wt. % of the one or more miscellaneous ingredients, based on the total weight of the compositions. In further embodiments, the compositions include about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. %, based on the total weight of the compositions.

pH

The pH of the composition can vary. Nonetheless, in various embodiments, a pH less than 7 (an acidic pH) is desirable. For example, the pH can be from about 3 to about 6.5, about 3 to about 6, about 3 to about 5.5, about 3.5 to about 6.5, about 3.5 to about 6, about 3.5 to about 5.5, about 4 to about 6.5, about 4 to about 6, about 4 to about 5.5, about 4.5 to about 6.5, about 4.5 to about 6, or about 4.5 to about 5.5, preferably about 4.5 to about 5.5.

### Embodiments of Leave-On Compositions

In various embodiments, the leave-on composition comprises, consists essentially of, or consists of:
(a) about 1 to about 10 wt. %, preferably about 2 to about 8, more preferably about 3 to about 6 wt. % of one or more silicones, preferably wherein one, more than one, or all of the silicones are selected from amino-functionalized silicones, preferably selected from amodimethicone, bis-hydroxy/methoxy amodimethicone, bis-cetearyl amodimethicone, bis(C13-15 alkoxy) PG amodimethicone, aminopropyl phenyl trimethicone, aminopropyl dimethicone, bis-amino PEG/PPG-41/3 aminoethyl PG-propyl dimethicone, or a mixture thereof, more preferably wherein the one or more silicones comprise or consist of amino-functionalized silicones selected from aminopropyl dimethicone, amodimethicone, or a combination thereof;
(b) about 0.1 to about 5 wt. %, preferably about 0.2 to about 4 wt. %, more preferably about 0.5 to about 3 wt. % of one or more film-forming polymers, preferably one or more polyurethane film-forming polymers, more preferably one or more polyurethane/polyether film forming polymers (also referred to a polyurethane/polyether nonionic associative polymeric thickeners) selected from PEG-240/HDI copolymer bis-decyltetradeceth-20 ether, PEG-150/stearyl alcohol/SMDI copolymer, PEG-150/decyl alcohol/SMDI copolymer, steareth-100/PEG-136/HDI copolymer, or a mixture thereof, in particular, PEG-240/HDI copolymer bis-decyltetradeceth-20 ether;
(c) about 60 to about 85 wt. %, preferably about 65 to about 80 wt. %, more preferably about 70 to about 80 wt. % of water;
(d) optionally, about 0.1 to about 12 wt. %, preferably about 1 to about 10 wt. %, more preferably about 2 to about 8 wt. % of one or more non-silicone-based fatty compounds, preferably wherein the one or more non-silicone-based fatty compounds are selected from oils, waxes, linear or branched alkanes, fatty esters, esters of fatty acids, esters of fatty alcohols, cetyl esters, triglycerides, or a mixture thereof, more preferably wherein the one or more non-silicone-based fatty compounds comprises or consists of one or more linear or branched alkanes, oils, or a mixture thereof;
(e) about 0.1 to about 5 wt. %, preferably about 0.2 to about 4 wt. %, more preferably about 0.2 to about 3 wt. % of one or more polysaccharides, preferably one or more polysaccharides selected from starches, modified starches, gums, modified gums, celluloses, modified celluloses, and a mixture thereof, more preferably wherein the one or more polysaccharides are selected from xanthan gum, gellan gum, sclerotium gum, guar gum and its derivatives, cellulose and its derivatives, and a mixture thereof, in particular sclerotium gum;
(f) optionally, about 0.1 to about 15 wt. %, more preferably about 1 to about 12 wt. %, more preferably about 2 to about 10 wt. % of one or more water soluble solvents, preferably wherein the one or more water soluble solvents are selected from glycerin, C₁-C₆ mono-alcohols, polyols (polyhydric alcohols), glycols, or a mixture thereof, more preferably wherein the one or more water soluble solvents are selected from glycerin, glycols (e.g., ethylene glycol, propylene glycol, butylene glycol, pentylene glycol, caprylyl glycol, etc.), or a combination thereof; and
(g) optionally, about 0.01 to about 10 wt. %, preferably about 0.1 to about 8 wt. %, more preferably about 1 to about 5 wt. % of one or more miscellaneous ingredients, preferably selected from preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, silica, particular materials, etc.), emollients, composition colorants, or a mixture thereof wherein all weight percentages of are based on a total weight of the leave-on composition.
   Application and Processing with Leave-On Composition

The leave-on composition can be applied to wet, damp, or dry hair. Nonetheless, the leave-on composition is typically applied to damp hair, for example, damp hair that has been treated with the conditioning composition shortly before application of the leave on composition. For example, the hair may have been treated with the conditioning composition within about 1 hour before application of the leave-on composition. Nonetheless, often the leave-on composition is applied more quickly after completing the treatment of the hair with the conditioning composition. For example, the leave-on composition may be applied to the hair within 30 minutes, within 15 minutes, within 10 minutes, within 5 minutes from completion of treating the hair with the conditioning composition.

The leave-on composition is distributed throughout the hair and allowed to remain on the hair. As the name indicates, the composition is a “leave-on” or “leave-in” product, meaning that the composition is not rinsed or washed away from the hair prior to drying and styling the hair. The composition remains on the hair for a long period of time (e.g., 12 hours, 24 hours, 2 days, 3 days, 5 days, 7 days, or longer. After the leave on composition has been applied to the hair, the hair is allowed to dry. The leave-on composition includes water, i.e., is an aqueous composition, and will therefore wet the hair upon application, assuming the hair is not already wet or damp upon application of the leave-on composition. The hair can be allowed to dry naturally but may be preferable to expedite the drying process using a blow dryer. A blow drying may be used, for example, with a brush to impart a straightening effect to the hair as it dries. For example, the brush can be used to pull and align the hair while the blow dryer heats and dries the hair. In any event, after the hair is dry, it can be subsequently treated with a thermal treatments (a heat treatment), for example, as discussed below.

### Thermal Heat Treatment

After application of the leave-on composition to the hair, the hair can be dried, for example, with a blow dryer. Alternatively, the hair can dry naturally. The thermal heat treatment typically occurs at a temperature of about 150 to about 300° C. In various embodiments, the heat treatment occurs at a temperature of about 150 to about 280° C., about 180 to about 300° C., about 180 to about 280° C., about 200 to about 300° C., about 200 to about 280° C., about 200 to about 260° C., about 200 to about 250° C., about 210 to about 300° C., about 210 to about 280° C., about, 180° C., about 190° C., about 200° C., about 210° C., about 220° C., about 230° C., about 240° C., about 250° C. or about 260° C.

In various embodiments, the heat treatment includes treating the hair with a hot iron, for example, a curling iron or a flat iron. The hot iron can be passed over the hair once or multiple times, for example, one or more passes, two or more passes, three or more passes, or four or more passes.

### Exemplary Methods

In various embodiments, the methods of the instant disclosure comprise, consist essentially of, or consist of:
(I) applying an alkaline composition having a pH of about 9 to about 12 to the hair, allowing the composition to remain on the hair for a period of time, preferably about 1 minute to about 60 minutes, and rinsing the alkaline composition from the hair; the alkaline composition comprising, consisting essentially of, or consisting of:
   (a) about 1 to about 15 wt. %, preferably about 5 to about 15 wt. %, more preferably about 5 to about 12 wt. % of one or more alkalizing agents comprising:
      (a)(i) about 2 to 15 wt. %, preferably about 2 to about 12 wt. %, more preferably about 5 to about 10 wt. % of one or more alkanolamines; and
      (a)(ii) optionally, about 1 to about 8 wt. %, preferably about 2 to about 5 wt. %, more preferably about 2 to about 6 wt. % of one or more additional alkalizing agents, preferably wherein at least one of the one or more additional alkalizing agents is selected from amino acids, hydroxide bases, or a combination thereof; and
   (b) about 50 to about 80 wt. %, preferably about 55 to about 75 wt. %, more preferably about 60 to about 70 wt. % of water;
   (c) about 1 to about 20 wt. %, preferably about 2 to about 15 wt. %, more preferably about 5 to about 12 wt. % of one or more non-silicone based fatty compounds comprising:
      (c)(i) about 1 to about 12 wt. %, preferably about 2 to about 10 wt. %, more preferably about 2 to about 8 wt. % of one or more fatty alcohols; and
      (c)(ii) optionally, about 0.1 to about 18 wt. %, preferably about 1 to about 12 wt. %, more preferably about 3 to about 10 wt. % of one or more additional fatty compounds;
   (d) about 0.1 to about 20 wt. %, preferably about 1 to about 15 wt. %, more preferably about 5 to about 15 wt. % of one or more water soluble solvents, preferably wherein the one or more water-soluble solvents are selected from glycerin, mono-alcohols (for example, C₁₋₁₀, C₁₋₈, or C₁₋₄ alcohols), polyols (polyhydric alcohols), glycols, and a mixture thereof;
   (e) about 0.1 to about 10 wt. %, preferably about 1 to about 10 wt. %, more preferably about 2 to about 8 wt. % of one or more nonionic surfactants; and
   (f) optionally, about 0.1 to about 8 wt. %, preferably about 0.2 to about 6 wt. %, more preferably about 0.3 to about 3 wt. % of one or more polysaccharide thickening agents;
      wherein all weight percentages of (i) are based on a total weight of the alkaline composition;
   (g) optionally, about 0.01 to about 8 wt. %, preferably about 0.05 to about 5 wt. %, more preferably about 0.1 to about 3 wt. % one or more silicones;
   (h) optionally, about 0.01 to about 10 wt. %, preferably about 0.1 to about 8 wt. %, more preferably about 1 to about 5 wt. % of one or more miscellaneous ingredients;
      wherein all percentages weight percentages of (I) are based on a total weight of the alkaline composition;
(II) within about 1 hour after rinsing the alkaline composition from the hair, applying a conditioning composition to the hair, allowing the conditioning composition to remain on the hair for a period of time, preferably for about 1 minute to about 60 minutes, and rinsing the conditioning composition from the hair; the conditioning composition comprising, consisting essentially of, or consisting of:
   (a) about 1 to about 5 wt. %, preferably about 1.5 to about 4, more preferably about 2 to about 4 wt. % of citric acid, a salt thereof, or a mixture thereof;
   (b) about 0.5 to about 5 wt. %, preferably about 1 to about 4, more preferably about 1 to about 3 wt. % of a cyclodextrin, derivative thereof, or combination thereof;
      wherein a combined total amount of the citric acid of (a) and the cyclodextrin of (b) is about 2 to about 8 wt. %; and
      wherein preferably the citric acid, salts thereof, or combination thereof of (a) and the cyclodextrin, derivative thereof, or combination thereof of (b) are individually combined with each other to form a composition in which the cyclodextrin, derivative thereof, or combination thereof of (b) is dissolved in the citric acid, salt thereof, or combination thereof of (a) before subsequently being combined with additional components of the hair-treatment composition;
   (c) about 1 to about 10 wt. %, preferably about 1 to about 8 wt. %, more preferably about 2 to about 6 wt. % of one or more cationic surfactants;
   (d) about 0.1 to about 10 wt. %, preferably about 0.5 to about 8, more preferably about 1 to about 5 of one or more nonionic surfactants or emulsifiers;
   (e) about 1 to about 15 wt. %, preferably about 2 to about 12, more preferably about 4 to about 10 wt. % of one or more fatty alcohols;
   (f) optionally, about 2 to about 18 wt. %, preferably about 2 to about 12 wt. %, more preferably about 3 to about 8 wt. % of one or more amino-functionalized silicones;
   (g) optionally, about 0.1 to about 14 wt. %, preferably about 1 to about 12 wt. %, more preferably about 2 to about 8 wt. % of;
   (h) about 60 to about 85 wt. %, preferably about 65 to about 80 wt. %, more preferably about 70 to about 80 wt. % of water;
   (i) optionally, about 0.1 to about 12 wt. %, preferably about 0.5 to about 10 wt. %, more preferably about 1 to about 6 wt. % of one or more non-silicone-based fatty compounds;
   (j) optionally, about 0.01 to about 5 wt. %, preferably about 0.1 to about 4 wt. %, more preferably about 0.1 to about 3 wt. % of one or more cationic conditioning polymers; and
   (k) optionally, about 0.01 to about 10 wt. %, preferably about 0.1 to about 8 wt. %, more preferably about 1 to about 5 wt. % of one or more miscellaneous ingredients;
      wherein all weight percentages of (II) are based on a total weight of the conditioning composition;
(III) within about 1 hour after rinsing the conditioning composition from the hair, applying a leave-on composition to the hair; the leave-on composition comprising, consisting essentially of, or consisting of:
   (a) about 1 to about 10 wt. %, preferably about 2 to about 8, more preferably about 3 to about 6 wt. % of one or more silicones, preferably one or more amino-functionalized silicones;
   (b) about 0.1 to about 5 wt. %, preferably about 0.2 to about 4 wt. %, more preferably about 0.5 to about 3 wt. % of one or more film-forming polymers, preferably one or more polyurethane film-forming polymers, more preferably one or more polyurethane/polyether film forming polymers (also referred to a polyurethane/polyether nonionic associative polymeric thickeners) selected from PEG-240/HDI copolymer bis-decyltetradeceth-20 ether, PEG-150/stearyl alcohol/SMDI copolymer, PEG-150/decyl alcohol/SMDI copolymer, steareth-100/PEG-136/HDI copolymer, or a mixture thereof, in particular, PEG-240/HDI copolymer bis-decyltetradeceth-20 ether;
   (c) about 60 to about 85 wt. %, preferably about 65 to about 80 wt. %, more preferably about 70 to about 80 wt. % of water;
   (d) optionally, about 0.1 to about 12 wt. %, preferably about 1 to about 10 wt. %, more preferably about 2 to about 8 wt. % of one or more non-silicone-based fatty compounds;
   (e) optionally, about 0.1 to about 5 wt. %, preferably about 0.2 to about 4 wt. %, more preferably about 0.2 to about 3 wt. % of one or more polysaccharide thickening agents;
   (f) optionally, about 0.1 to about 15 wt. %, more preferably about 1 to about 12 wt. %, more preferably about 2 to about 10 wt. % of one or more water soluble solvents;
   (g) optionally, about 0.01 to about 10 wt. %, preferably about 0.1 to about 8 wt. %, more preferably about 1 to about 5 wt. % of one or more miscellaneous ingredients;
      wherein all weight percentages of (III) are based on a total weight of the leave-on composition;
   (IV) optionally, without removing the leave-on composition, drying the hair, for example, with a blow dryer;
   (V) after drying the hair and without removing the leave-on composition from the hair, subjecting the hair to a thermal treatment, preferably treating the hair with a hot iron, wherein the hot iron is preferably a flat iron, wherein the temperature of the hot iron is preferably from about 150 to about 280° C., preferably a temperature of about 150 to about 220° C., more preferably at a temperature of about 160 to about 200° C.

Implementation of the present disclosure is provided by way of the following examples. The examples serve to illustrate the technology without being limiting in nature.

### Example 1

### Alkaline Composition

| | | (i) |
|---|---|---|
| (a) | ETHANOLAMINE, | 10.3 |
| | SODIUM HYDROXIDE, | |
| | AND TAURINE | |
| (c) | CETEARYL ALCOHOL | 2.5 |
| | C13-16 ISOALKANE | 5 |
| (d) | PENTYLENE GLYCOL | 10 |
| | AND ETHANOL | |
| (e) | LAURETH-23, LAURETH-4, | 4 |
| | STEARETH-20, AND/OR | |
| | STEARETH-2 | |
| (f) | HYDROXYPROPYL GUAR | 1.5 |
| (g) | DIMETHICONE | 0.1 |
| (h) | MISCELLANEOUS | ≤5 |
| (b) | WATER | QS |

| | |
|---|---|
| pH | 10.5-10.9 |

### Example 2

### Conditioner Composition

| | | (ii) |
|---|---|---|
| (a) | CITRIC ACID | 2.5 |
| (b) | CYCLODEXTRIN | 1.5 |
| Total (a) + (b) | | 4 |
| Ratio of (a):(b) | | 1.7 |
| (c) | BEHENTRIMONIUM CHLORIDE | 2.4 |
| | CETRIMONIUM CHLORIDE | 0.05 |
| (d) | TRIDECETH-5, TRIDECETH-10, | 1.4 |
| | TRIDECETH-6, GLYCERYL | |
| | LINOLENATE, GLYCERYL | |
| | OLEATE, GLYCERYL | |
| | LINOLEATE, AND/OR | |
| | SAFFLOWER GLUCOSIDE | |
| (e) | CETEARYL ALCOHOL | 6 |
| (f) | AMODIMETHICONE | 4.8 |
| (g) | GLYCERIN, BUTYLENE | 3.4 |
| | GLYCOL, AND CAPRYLYL | |
| | GLYCOL | |
| | ISOPROPYL ALCOHOL | |
| (i) | CETYL ESTERS | 1.5 |
| | HELIANTHUS ANNUUS | 0.01 |
| | (SUNFLOWER) SEED OIL | |
| (j) | HYDROXYPROPYLTRIMONIUM | 0.1 |
| | HYDROLYZED WHEAT | |
| | PROTEIN | |
| (k) | MISCELLANEOUS* | ≤5 |
| | (Fillers, pH adjusters, | |
| | preservatives, botanical extract, | |
| | fragrances, buffers, stabilizers, | |
| | vitamins, and/or emollients, etc.) | |
| (h) | WATER | QS |

| | |
|---|---|
| pH | 3.3 |
| Viscosity | 134,000 cP |

*Styrax benzoin gum, boron nitride, xylose, acetic acid, benzoic acid, iris Florentina root extract, fragrance phenoxyethanol, sodium citrate, BHT, sodium hydroxide, tocopherol.

### Example 3

### Leave-On Composition

| | | (iii) |
|---|---|---|
| (a) | AMINOPROPYL | 4.9 |
| | DIMETHICONE | |
| (b) | PEG-240/HDI COPOLYMER | 0.9 |
| | BIS-DECYLTETRADECETH-20 | |
| | ETHER | |
| (d) | UNDECANE, ISODODECANE, | 8 |
| | ISONONYL ISONONANOATE | |
| | TRIDECANE, AND | |
| | HELIANTHUS ANNUUS | |
| | (SUNFLOWER) SEED OIL | |
| (e) | SCLEROTIUM GUM | 0.5 |
| (f) | BUTYLENE GLYCOL AND | 6.2 |
| | CAPRYLYL GLYCOL | |
| (g) | MISCELLANEOUS * | ≤5 |
| (c) | WATER | QS |

| | |
|---|---|
| pH | 4.5-5.5 |

* For example, preservative, fragrances, pH adjustors, buffers, chelating agent composition colorants, vitamins, amino acids, peptides, proteins, protein isolates or hydrolysates, salts, etc. (for example potassium laurate, citric acid, sodium citrate, tocopherol, etc.)

The foregoing description illustrates and describes the disclosure. Additionally, the disclosure shows and describes only the preferred embodiments. However, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions may be modified in all instances by the term “about,” meaning within +/−5% of the indicated number.

All percentages, parts and ratios herein are based upon the total weight of the compositions of the present invention, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered both a nonionic surfactant or emulsifier and a fatty compound. If a particular composition includes both a nonionic surfactant or emulsifier and a fatty compound, a single compound will serve as only the nonionic surfactant or emulsifier or only as the fatty compound (the single compound does not simultaneously serve as both the nonionic surfactant or emulsifier and the fatty component).

A “rinse-off” product refers to a composition that is rinsed and/or washed from the hair with water either after or during the application of the composition onto the hair, and before drying and/or styling the hair. At least a portion of the composition is removed from the hair during the rinsing and/or washing.

A “leave-on” product refers to a composition that is not rinsed and/or washed from the hair after or during application of the composition onto the hair. The composition remains on the hair during drying and/or throughout styling.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The composition of the instant case optionally include one or more surfactants and/or emulsifiers, for example, one or more nonionic, anionic, cationic, and/or amphoteric/zwitterionic surfactants. The term “surfactants” and “emulsifiers” include salts of the surfactants and emulsifiers even if not explicitly stated. In other words, whenever the disclosure refers to a surfactant or emulsifier, it is intended that salts are also encompassed to the extent such salts exist, even though the specification may not specifically refer to a salt (or may not refer to a salt in every instance throughout the disclosure), for example, by using language such as “a salt thereof” or “salts thereof.” Sodium and potassium are common cations that form salts with surfactants and emulsifiers. However, additional cations such as ammonium ions, or alkanolammonium ions such as monoethanolammonium or triethanolammonium ions, may also form salts of surfactants.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material.

All components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A method for treating hair comprising:
(i) applying an alkaline composition having a pH of about 9 to about 12 to the hair, allowing the composition to remain on the hair for a period of time, and rinsing the alkaline composition from the hair; the alkaline composition comprising:
(a) one or more alkalizing agents comprising:
(a)(i) one or more alkanolamines; and
(a)(ii) optionally, one or more additional alkalizing agents; and
(b) water;
(ii) after rinsing the alkaline composition from the hair, applying a conditioning composition to the hair, allowing the conditioning composition to remain on the hair for a period of time, and rinsing the conditioning composition from the hair; the conditioning composition comprising:
(a) citric acid, a salt thereof, or a combination thereof;
(b) cyclodextrin, a derivative thereof, or a combination thereof;
wherein a combined total amount of the citric acid of (a) and the cyclodextrin of (b) is about 2 to about 8 wt. %; and
(c) one or more cationic surfactants; and
(h) water; and
(iii) after rinsing the conditioning composition from the hair, applying a leave-on composition to the hair; the leave-on hair composition comprising:
(a) one or more silicones;
(b) one or more film-forming polymers; and
(c) water.

2. The method of claim 1, wherein the alkaline composition remains on the hair for about 1 minute to about 30 minutes before being rinsed from the hair.

3. The method of claim 1, wherein the conditioning composition has a pH of about 2 to about 6, preferably about 2 to about 5.

4. The method of claim 1, wherein the conditioning composition remains on the hair for about 1 minute to about 30 minutes before being rinsed from the hair.

5. The method of claim 1, wherein the leave-on composition has a pH of about 2 to about 6, preferably about 2 to about 5.

6. The method of claim 1, further comprising:
(iv) drying the hair and styling the hair.

7. The method of claim 6, wherein the hair is styled with a hot iron at a temperature of about 150 to about 280° C.

8. The method of claim 1, wherein the alkaline composition further comprises:
(c) about 2 to about 20 wt. % of one or more fatty compounds comprising:
(c)(i) about 2 to about 12 wt. % of one or more fatty alcohols; and
(c)(ii) optionally, about 1 to about 10 wt. % of one or more additional fatty compounds;
wherein all weight percentages are based on a total weight of the alkaline composition.

9. The method of claim 1, wherein the alkaline composition further comprises:
(d) about 1 to about 20 wt. % of one or more water soluble solvents; and/or
(e) about 1 to about 8 wt. % of one or more nonionic surfactants or emulsifiers; and/or
(f) one or more polysaccharide thickening agents;
wherein all weight percentages are based on a total weight of the alkaline composition.

10. The method of claim 1, wherein the conditioning composition further comprises:
(d) about 0.1 to about 5 wt. % of one or more nonionic surfactants or emulsifiers;
wherein all weight percentages are based on a total weight of the conditioning composition.

11. The method of claim 1, wherein the conditioning composition further comprises:
(e) about 1 to about 10 wt. % of one or more fatty alcohols;
(f) optionally, one or more amino-functionalized silicones; and
(g) optionally, one or more water soluble solvents;
wherein all weight percentages are based on a total weight of the conditioning composition.

12. The method of claim 1, wherein the one or more silicones of the leave-on composition are selected from one or more amino-functionalized silicones.

13. The method of claim 1, wherein the one or more film-forming polymers of the leave-on composition are selected from nonionic associative polymeric thickeners

14. The method of claim 1, wherein the leave-on composition further comprises:
(d) about 1 to about 15 wt. % of one or more fatty compounds; and/or
(e) about 0.1 to about 8 wt. % of one or more polysaccharide thickening agents; and/or
(f) about 1 to about 15 wt. % of one or more water soluble solvents;
wherein all weight percentages are based on a total weight of the leave-on composition.

15. The method of claim 1, wherein the alkaline composition comprises:
(a) about 2 to about 15 wt. % of one or more alkalizing agents comprising:
(a)(i) about 2 to 15 wt. % of one or more alkanolamines; and
(a)(ii) optionally, about 1 to about 8 wt. % of one or more additional alkalizing agents; and
(b) about 50 to about 80 wt. % of water;
(c) about 2 to about 20 wt. % of one or more fatty compounds comprising:
(c)(i) about 2 to about 12 wt. % of one or more fatty alcohols; and
(c)(ii) optionally, about 1 to about 10 wt. % of one or more additional fatty compounds;
(d) about 1 to about 20 wt. % of one or more water soluble solvents;
(e) about 1 to about 8 wt. % of one or more nonionic surfactants or emulsifiers; and
(f) about 0.1 to about 8 wt. % of one or more polysaccharide thickening agents;
wherein all weight percentages are based on a total weight of the alkaline composition.

16. The method of claim 1, wherein the conditioning composition:
(a) about 1 to about 5 wt. % of citric acid, a salt thereof, or a derivative thereof;
(b) about 1 to about 5 wt. % of a cyclodextrin, a derivative thereof, or a salt thereof;
wherein a combined total amount of (a) and (b) is about 2 to about 8 wt. %;
(c) about 1 to about 10 wt. % of one or more cationic surfactants;
(d) about 0.1 to about 5 wt. % of one or more nonionic surfactants or emulsifiers;
(e) about 1 to about 10 wt. % of one or more fatty alcohols;
(f) about 2 to about 18 wt. % of one or more amino-functionalized silicones;
(g) optionally, one or more water-soluble solvents;
(h) about 60 to about 85 wt. % of water;
wherein all weight percentages are based on a total weight of the conditioning composition.

17. The method of claim 1, wherein the leave-on composition comprises:
(a) about 1 to about 12 wt. % of one or more amino-functionalized silicones;
(b) about 0.1 to about 3 wt. % of one or more nonionic associative polymeric thickeners;
(c) about 65 to about 85 wt. % of water;
(d) about 1 to about 15 wt. % of one or more fatty compounds;
(e) about 0.1 to about 8 wt. % of one or more polysaccharide thickening agents; and
(f) about 1 to about 15 wt. % of one or more water soluble solvents;
wherein all weight percentages are based on a total weight of the leave-on composition.

18. A method for treating hair comprising:
(i) applying an alkaline composition having a pH of about 9 to about 12 to the hair, allowing the composition to remain on the hair for a period of time, and rinsing the alkaline composition from the hair; the alkaline composition comprising:
(a) about 2 to about 15 wt. % of one or more alkalizing agents comprising:
(a)(i) about 2 to 15 wt. % of one or more alkanolamines; and
(a)(ii) optionally, about 1 to about 8 wt. % of one or more additional alkalizing agents; and
(b) about 50 to about 80 wt. % of water;
(c) about 2 to about 20 wt. % of one or more fatty compounds comprising:
(c)(i) about 2 to about 12 wt. % of one or more fatty alcohols; and
(c)(ii) optionally, about 1 to about 10 wt. % of one or more additional fatty compounds;
(d) about 1 to about 20 wt. % of one or more water soluble solvents;
(e) about 1 to about 8 wt. % of one or more nonionic surfactants or emulsifiers; and
(f) about 0.1 to about 8 wt. % of one or more polysaccharide thickening agents;
wherein all weight percentages of (i) are based on a total weight of the alkaline composition;
(ii) after rinsing the alkaline composition from the hair, applying a conditioning composition to the hair, allowing the conditioning composition to remain on the hair for a period of time, and rinsing the conditioning composition from the hair; the conditioning composition comprising:
(a) about 1 to about 5 wt. % of citric acid, a salt thereof, or a combination thereof;
(b) about 1 to about 5 wt. % of a cyclodextrin, a derivative thereof, or a combination thereof;
wherein a combined total amount of (a) and (b) is about 2 to about 8 wt. %;
(c) about 1 to about 10 wt. % of one or more cationic surfactants;
(d) about 0.1 to about 5 wt. % of one or more nonionic surfactants or emulsifiers;
(e) about 1 to about 10 wt. % of one or more fatty alcohols;
(f) about 2 to about 18 wt. % of one or more amino-functionalized silicones;
(g) optionally, one or more water-soluble solvents;
(h) about 60 to about 85 wt. % of water;
wherein all weight percentages of (ii) are based on a total weight of the conditioning composition;
(iii) after rinsing the conditioning composition from the hair, applying a leave-on composition to the hair; the leave-on composition comprising:
(a) about 1 to about 12 wt. % of one or more amino-functionalized silicones;
(b) about 0.1 to about 3 wt. % of one or more nonionic associative polymeric thickeners;
(c) about 65 to about 85 wt. % of water;
(d) about 1 to about 15 wt. % of one or more fatty compounds;
(e) about 0.1 to about 8 wt. % of one or more polysaccharide thickening agents; and
(f) about 1 to about 15 wt. % of one or more water soluble solvents;
wherein all weight percentages of (iii) are based on a total weight of the leave-on composition.

19. The method of claim 18, further comprising:
(iv) drying the hair and styling the hair.

20. The method of claim 19, wherein the hair is styled with a hot iron at a temperature of about 150 to about 280° C.
